(19) United States
(12) Patent Application Publication
(10) Pub. No.: US 2018/0221329 A1
(43) Pub. Date: Aug. 9, 2018

(54) CIS- AND TRANS-SUFFRUTICOSOL D AS THERAPEUTIC AGENTS

(71) Applicant: MIDDLE TENNESSEE STATE UNIVERSITY, Murfreesboro, TN (US)

(72) Inventors: Ying Gao, Murfreesboro, TN (US); Elliot Altman, Rockvale, TN (US)

(73) Assignee: MIDDLE TENNESSEE STATE UNIVERSITY, Murfreesboro, TN (US)

(21) Appl. No.: 15/747,630

(22) PCT Filed: Jul. 28, 2016
(86) PCT No.: PCT/US16/44534

§ 371 Date: Jan. 26, 2018

Related U.S. Application Data
(60) Provisional application No. 62/197,909, filed on Jul. 28, 2015.

(51) Int. Cl. A61K 31/343; A61P 35/04; A61K 36/65; A61K 45/06

(52) U.S. Cl. CPC A61K 31/343; A61K 45/06; A61K 36/65; A61P 35/04

(57) ABSTRACT

The cis- and trans-isomers of the plant-derived compound suffruticosol D are shown to have anticancer properties. Therapeutic and prophylactic compositions that contain cis- or trans-suffruticosol D, as well as methods of making and using said compositions, are provided. The cis- or /ra«s-suffruticosol D can be used in purified form or as a plant extract.

This application claims the benefit of U.S. Provisional Application Ser. No. 62/197,909, filed Jul. 28, 2015, which is incorporated herein by reference in its entirety.

### BACKGROUND

Current cancer medications are costly and often cause serious side effects. The US National Cancer Institute began investigating anti-tumor plant extracts in the 1960s (Cai et al., 2004, 74(17): 2157-2184; Monks et al., 2002, 40(8): 603-616), and the premise that natural compounds obtained from therapeutic plants could produce anti-cancer medications has since then been of great research interest. Traditional Chinese Medicines (TCMs) using dried plants or plant extracts have provided low cost dietary and pharmaceutical therapies for thousands of years, and experimental and clinical studies have proven that more than 400 plant species used in TCMs as anti-cancer herbal medications are significantly effective in the prevention or treatment of various cancers (Ji et al., 1999, Pharmacological Action and Application of Anticancer Traditional Chinese Medicines, Heilongjiang Science and Technology Publishing House, Ha'erbin, China; Xu et al., 2000, Coloured Illustrations of Antitumor Chinese Traditional And Herbal Drugs (2nd) Fujian Science and Technology Publishing House, Fuzhou, China; Bo et al., 2002, A Selection of the Illustrated Chinese Anti-Cancer Herbal Medicines. Shanghai Science and Technology Literature Press, Shanghai; Cai et al., 2004, 74(17): 2157-2184; Parekh et al., 2009, Mol. Cancer, 8(1):21). However, much work remains to be done to determine the effectiveness of the individual compounds present in the TCMs.

Paeonia suffruticosa, or Paeoniaceae, is a widely utilized Chinese medicinal plant within the Paeonia genus. This genus comprises approximately 35 species that are classified into three groups: Oneapia, Paeonia, and Moutan (He et al., 2010, Chem. Pharmaceut. Bull. 58(6):843-847). The Cortex Moutan (root cortex) of Paeonia has been recorded by China's Pharmacopoeia as a significant source of herbal medicine (Chinese Pharmacopoeia Commission, 2010). Extracts of Paeonia have been shown to possess cytotoxic, antitumor, anti-inflammatory and anti-oxidative activities (He et al., 2010, Chem. Biodiversity 7(4), 805-838). Previous photochemical research on Paeonia identified more than 260 bioactive compounds, including phenols, monoterpenoidglucosides, paeonols, flavonoids, tannins, steroids, triterpenoids and stilbenes A more recent study showed that the seeds of Paeonia contain considerable quantities of stilbenes compared to the other compounds (He et al., 2013, J. Liq. Chromatog. Related Technolog. 36(12):1708-1724).

Stilbenes, widely found in plants, are a class of polyphenols that contain a 1,2-diphenylethylene nucleus in their structure. Stilbenes have aroused considerable interest due to their anti-tumor, anti-steroidal, anti-mutagenic, anti-oxidative and anti-inflammatory bioactivities (Hussain et al., 2009, BMC Cell Biol., 10(1):30; Sangjun et al., 2009, Toxicol. Lett. 186(2):115-122; Savio et al., 2009, Int. J. Biochem. Cell Biol. 41(12):2493-2502; Simoni et al., 2009, Bioorg. Med. Chem. 17(2), 512-522. Cai et al., 2011, Biol. Pharmaceut. Bull. 34(9):1501-1507), Yuk et al., 2013, Food Chem. Toxicol. 55:144-149). One well-known example of the stilbenes is resveratrol, and its anti-tumor activity has been intensively studied.

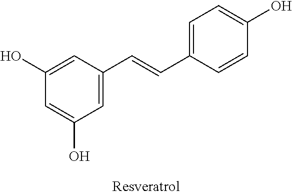 [SMILES: Oc1ccc(/C=C/c2cc(O)cc(O)c2)cc1]

Several in vivo and in vitro studies have shown that resveratrol inhibits the growth of cancer cells and affects various molecular targets associated with cancer progression such as the Wnt signaling pathway, nuclear factor-kappa B (NF-κB), and the MAPK/ERK pathway in different types of cancer (Shukla et al., Ann NY Acad Sci 1215: 1-8, 2011; Whitlock et al., Nutr Cancer 64: 493-502, 2012).

### SUMMARY OF THE INVENTION

The invention identifies cis-suffruticosol D and trans-suffruticosol D as novel therapeutic agents. In one aspect, the invention provides a method for treating or preventing cancer or a precancerous condition in a subject, which method includes administering to the subject a composition comprising an effective amount of cis-suffruticosol D, trans-suffruticosol D, or a combination thereof. The cancer or precancerous condition can involve any tissue or organ, such as bone, brain, breast, cervix, larynx, lung, pancreas, prostate, skin, spine, stomach, uterus or blood. The cancer can be a bone cancer, brain cancer, breast cancer, cervical cancer, cancer of the larynx, lung cancer, pancreatic cancer, prostate cancer, skin cancer, cancer of the spine, stomach cancer, uterine cancer, or a blood cancer. The cancer can be a metastatic cancer.

In another aspect, the invention provides a method for inhibiting the growth of a tumor in a subject, which method includes administering to the subject a composition comprising an effective amount of cis-suffruticosol D, trans-suffruticosol D, or a combination thereof. The tumor may include a solid tumor present in the bone, brain, breast, cervix, larynx, lung, pancreas, prostate, skin, spine, stomach, or uterus of the subject. The tumor may be a fast growing tumor.

The composition may include an extract prepared from Paeonia suffruticosa seeds. Optionally, the composition further includes a pharmaceutically acceptable carrier. In some embodiments, the composition may further include a non-naturally occurring therapeutic agent, such as at cytokine, a chemokine, a therapeutic antibody, an adjuvant, an antioxidant, or a chemotherapeutic agent.

In another aspect, the invention includes cis-suffruticosol D, trans-suffruticosol D, or a combination thereof for use as a therapeutic agent, including use in the treatment of cancer or a precancerous condition, or for use in inhibiting the growth of a tumor. Use of cis-suffruticosol D, trans-suffruticosol D, or a combination thereof for preparation of a medicament for the treatment of cancer or a precancerous condition, or for inhibiting the growth of a tumor, is also included in the invention.

In another aspect, the invention includes a plant extract that includes cis-suffruticosol D, trans-suffruticosol D, or a combination thereof, for use as a therapeutic agent, including use in the treatment of cancer or a precancerous condition, or for use in inhibiting the growth of a tumor. Use of a plant extract including cis-suffruticosol D, trans-suffruticosol D, or a combination thereof for preparation of a medicament for the treatment of cancer or a precancerous condition, or for inhibiting the growth of a tumor, is also included in the invention. In some embodiments, the plant extract is prepared from Paeonia suffruticosa seeds.

### BRIEF DESCRIPTION OF THE FIGURES

FIG. 1 depicts chemical structures of cis- and trans-suffruticosol D.

FIGS. 2A-E depict induction of apoptosis by cis- and trans-suffruticosol D in A549 cells. After 24 h treatment by cis- or trans-suffruticosol D, A549 cells were stained with Annexin V/7-amino-actinomycin D (7-AAD) and the percentage of apoptotic cells, as measured by fluorescence intensity, was assessed by flow cytometry. Cells treated with the vehicle, 1% dimethylsulfoxide, DMSO, only served as a negative control.

FIG. 2A shows annexin V/7-AAD double staining of A549 cells treated with various concentrations of trans-suffruticosol D. The x-axis represents annexin V and the y-axis represents 7-AAD.

FIG. 2B shows annexin V/7-AAD double staining of A549 cells treated with various concentrations of cis-suffruticosol D. The x-axis represents annexin V and the y-axis represents 7-AAD.

FIG. 2C shows annexin V/7-AAD double staining of A549 cells treated with the vehicle only. The x-axis represents annexin V and the y-axis represents 7-AAD.

FIG. 2D shows the percentage of apoptotic cells induced by cis- or trans-suffruticosol D (n=3).

FIG. 2E shows the effect of cis- or trans-suffruticosol D on key regulatory proteins of apoptosis, BH3 interacting-domain death agonist (BID); death receptor 6 (DR6), also known as Tumor necrosis factor receptor superfamily member 21 (TNFRSF21); and the cyclin dependent kinase inhibitor p27 (n=4). F, Effect of cis- or trans-suffruticosol D on key regulatory proteins of apoptosis, Hsp60, Hsp70, survivin, and X-linked inhibitor of apoptosis protein (XIAP) (n=4).

FIGS. 3A and 3B depict induction of oxidative stress by cis- and trans-suffruticosol D in A549 cells. A549 cells were treated with various concentrations of cis- or trans-suffruticosol D for 24 h, then stained with Hoechst and dihydroethidium (DHE) dye. Cells treated with Doxorubicin served as a positive control, and cells treated with vehicle only served as a negative control. The reactive oxygen species (ROS) levels were measured by the fluorescent intensity of DHE that was converted to ethidium bromide.

FIG. 3A shows fluorescent cell images by the high content screening (HCS) reader.

FIG. 3B shows ROS levels in A549 cells treated with various concentrations of cis- or trans-suffruticosol D.

FIGS. 4A and 4B depict cell motility changes induced by cis- and trans-suffruticosol D in A549 cells. A549-GFP cells were seeded in 96-well plate with a monolayer of fluorescent beads. After treatment with cis- or trans-suffruticosol D for 18 h, individual cell movement was evaluated by measuring the fluorescent track area. Cells treated with serum free medium served as a negative control and cells treated with medium containing 10% serum served as a positive control.

FIG. 4A shows the fluorescent track area showing the movement of the cells.

FIG. 4B shows measurement of the cell track areas of cells treated with various concentrations of cis- or trans-suffruticosol D (n=4).

FIGS. 5A-D depict multi-parameter analysis of cytotoxicity induced by cis- and trans-suffruticosol D in A549 cells. A549 cells were treated with various concentrations of cis- or trans-suffruticosol D for 24 h, then stained with three dyes simultaneously (Hoechst, cell permeability dye, and mitochondrial membrane potential dye). Cells treated with vehicle only served as a negative control and cells treated with 10 μM valinomycin served as a positive control.

FIG. 5A shows fluorescent cell images by HCS reader.

FIG. 5B shows evaluation of nuclear size of cells treated with cis- or trans-suffruticosol D (n=3).

FIG. 5C shows evaluation of cell permeability of cells treated with cis- or trans-suffruticosol D (n=3).

FIG. 5D shows evaluation of mitochondrial membrane potential of cells treated with cis- or trans-suffruticosol D (n=3).

FIGS. 6A and 6B depict inhibition of NF-κB translocation by cis- and trans-suffruticosol D in A549 cells. A549 cells were treated with various concentrations of cis- or trans-suffruticosol D for 4 h and then stimulated with 25 ng/mL TNF-α for 30 min. Cells treated with TNF-α alone or the vehicle only served as controls. The NF-κB translocation index was measured by the fluorescent intensity difference between the nucleus and cytoplasm.

FIG. 6A shows Western blotting analysis of the expression of phosphorylated-NF-κB p65 and total NF-κB p65.

FIG. 6B shows fluorescent cell images by the HCS reader. C, Evaluation of the NF-κB translocation index in A549 cells n=3).

FIG. 7 depicts a proposed cytotoxicity mechanism for cis- and trans-suffruticosol D.

### DESCRIPTION OF ILLUSTRATIVE EMBODIMENTS

The present invention provides compounds, compositions and methods relating to cis-suffruticosol D, including modifications, derivatives and conjugates thereof, and its use as a prophylactic or therapeutic agent, for example, to prevent or treat the cancers, precancerous conditions, or growth of tumors. cis-Suffruticosol D can be isolated or extracted from naturally occurring sources or can be chemically or enzymatically synthesized. cis-Suffruticosol D can be administered alone or in combination with other therapeutics via a variety of routes of administration.

Also provided are compounds, compositions and methods relating to trans-suffruticosol D, including modifications, derivatives and conjugates thereof, and its use as a prophylactic or therapeutic agent, for example, to prevent or treat the cancers, precancerous conditions, or growth of tumors. trans-Suffruticosol D can be isolated or extracted from naturally occurring sources or can be chemically or enzymatically synthesized. trans-Suffruticosol D can be administered alone or in combination with other therapeutics via a variety of routes of administration.

Also provided are compositions and methods that employ a combination of cis- and trans-suffruticosol D, including modifications, derivatives and conjugates thereof, and their use as prophylactic or therapeutic agents, for example, to prevent or treat the cancers, precancerous conditions, or growth of tumors. The combination of cis- and trans-suffruticosol D can be obtained from a plant extract and can be in the form of a racemic mixture; alternatively, isolated, purified and/or chemically or enzymatically synthesized cis- and trans-suffruticosol D can be combined to form the combination. The combination can be administered alone or in combination with other therapeutics via a variety of routes of administration.

We have found that both cis- and trans-suffruticosol D have promising antitumor activities. Both compounds selectively inhibited the growth of various cancer cells, induced apoptosis in cancer cells, as well as inhibited cancer cell mobility. Our findings suggest that both cis- and trans-suffruticosol D have promising chemotherapeutic potential for treating cancer.

cis- and Trans-Suffruticosol D

cis-Suffruticosol D and trans-suffruticosol D are resveratrol trimers and have similar structures, varying only in the position of groups with respect to an ethene double bond. The structure is shown below:

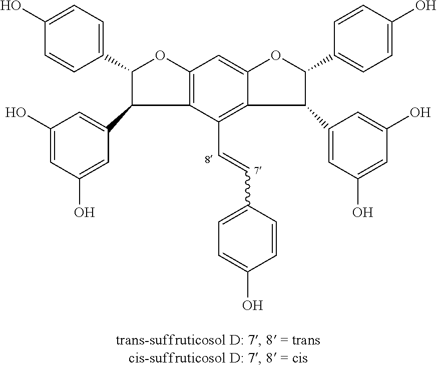 [SMILES: Oc1ccc(C=Cc2c3c(cc4c2[C@@H](c2cc(O)cc(O)c2)[C@H](c2ccc(O)cc2)O4)O[C@H](c2ccc(O)cc2)[C@@H]3c2cc(O)cc(O)c2)cc1]

The invention includes purified and partially purified forms of cis-suffruticosol D and trans-suffruticosol D, as well as crude plant extracts that contain cis-suffruticosol D and/or trans-suffruticosol D.

Also included in the invention are synthetic derivatives of cis-suffruticosol D and trans-suffruticosol D. Derivatives include, but are not limited to, alkylated (e.g., methylated), hydroxylated, sulfated and amino derivatives of cis-suffruticosol D and trans-suffruticosol D.

Not all naturally occurring stilbenes or their derivatives have anti-cancer or anti-tumor activity. See, for example Kim et al., Arch. Pharm. Res. 25(3) 293-299 (2002), and Kim et al., Biosci. Biotechnol. Biochem., 66(9): 1990-1993 (2002). It is known that the biological activities of cis- and trans-isomers of naturally occurring compounds may differ (see, e.g., Zhao et al., Acta Phys. Chim. Sin. 29 (1), 43-54 (2013); Anisimova et al., Chem. Cent. J. 5:88 (2011); Pettit et al., J. Nat. Prod., 72:1637-1642 (2009)). Thus, it was not known in advance whether cis-suffruticosol D and/or trans-suffruticosol D would exhibit the anti-cancer and anti-tumor properties described in Example 1.

Isolation or Synthesis of cis- and trans-Suffruticosol D

cis-Suffruticosol D and/or trans-suffruticosol D can be extracted and/or isolated from peony plants (genus Paeonia), including but not limited to Paeonia suffruticosa, Paeonia lactiflora, or Paeonia anamola. Any convenient plant part can serve as a source of cis-suffruticosol D or trans-suffruticosol D including, without limitation, the seeds, leaves, stems, roots, or flowers. In a preferred embodiment, the cis-suffruticosol D and/or trans-suffruticosol D is obtained from a root or seed extract of Paeonia suffruticosa or Paeonia lactiflora.

Additionally, trans-suffruticosol D can be photooxidatively transformed to cis-suffruticosol D. For example, trans-suffruticosol D can be photooxidized to cis-suffruticosol D for a time period of 2 hours, 4 hours, or 6 hours. Photooxidation can take place with light source such as a fluorescent lamp and optionally a photoactivating compound.

It is expected that cis-suffruticosol D and/or trans-suffruticosol D can be enzymatically synthesized using the appropriate plant enzymes. In one embodiment, resveratrol may be a starting material. Optionally, a stilbene synthase can be used, and additional co-factors can also be introduced, including but not limited to, malonyl-coenzyme A (CoA) and p-coumaroyl-CoA (Aggarwal et al., 2004, Anticancer Res. 24:2783-2840).

### Pharmaceutical Compositions

The present invention also provides a pharmaceutical composition that includes, as an active agent, cis-suffruticosol D and/or trans-suffruticosol D, or a synthetic derivative thereof and a pharmaceutically acceptable carrier. The active agent is formulated in a pharmaceutical composition and then, in accordance with the method of the invention, administered to a mammal, such as a human patient, in any of a variety of forms adapted to the chosen route of administration. The formulations include those suitable for oral, rectal, vaginal, topical, nasal, ophthalmic or parenteral (including subcutaneous, intramuscular, intraperitoneal, intratumoral, and intravenous) administration.

The pharmaceutically acceptable carrier can include, for example, an excipient, a diluent, a solvent, an accessory ingredient, a stabilizer, a protein carrier, or a biological compound. Nonlimiting examples of a protein carrier includes keyhole limpet hemocyanin (KLH), bovine serum albumin (BSA), ovalbumin, or the like. Nonlimiting examples of a biological compound which can serve as a carrier include a glycosaminoglycan, a proteoglycan, and albumin. The carrier can be a synthetic compound, such as dimethyl sulfoxide or a synthetic polymer, such as a polyalkyleneglycol. Ovalbumin, human serum albumin, other proteins, polyethylene glycol, or the like can be employed as the carrier. In a preferred embodiment, the pharmaceutically acceptable carrier includes at least one compound that is not naturally occurring or a product of nature.

In some embodiments, the active agent cis-suffruticosol D and/or trans-suffruticosol D, or a synthetic derivative thereof, is formulated in combination with one or more additional active agents, such an anticancer, antiangiogenic, or chemotherapeutic compound. In some embodiments, the pharmaceutical composition of the invention contains a first active agent that includes cis-suffruticosol D and/or trans-suffruticosol D, or a synthetic derivative thereof, and a second active agent that can include one or more of, for example, an anticancer agent, antiangiogenic agent, a chemopreventive agent, an anti-inflammatory agent, a cytokine, a chemokine, a therapeutic antibody, an immunogen, an antigen, an adjuvant, or an antioxidant, an immunomodulatory compound, an analgesic, a biologic compound, an antineoplastic agent, or a chemotherapeutic agent. A natural product, such as a plant product, or derivative thereof, having anticancer activity can, for example, be included in the pharmaceutical composition as a second active agent. See, e.g., Prakash et al., Am J. Pharmacolog. Sci., 1(6):104-115, for examples of plant compounds with anticancer activity. In an exemplary embodiment, cis-suffruticosol D and/or trans-suffruticosol D can be co-administered with cis-gnetin H and/or trans-gnetin H (PCT Publ. WO2016/049012; Park et al., J. Ethnopharmacol. 2016 May 16. pii: S0378-8741(16)30315-4. doi: 10.1016/j.jep.2016.05.042). In another exemplary embodiment, cis-suffruticosol D and/or trans-suffruticosol D can be co-administered with 2-dodecyl-6-methoxycyclohexa-2,5-diene-1,4-dione (PCT Publ. WO2016/094554; Gao et al., 2015, Oncotarget 6(27):24304-19). More generally, any therapeutic agent can be included as additional active agent. The action of the additional active agent in the combination therapy can be cumulative to the cis-suffruticosol D and/or trans-suffruticosol D or it can be complementary, for example to manage side effects or other aspects of the patient's medical condition.

A pharmaceutical composition of the invention may include at least one compound that is not naturally occurring or a product of nature. In a particularly preferred embodiment, the pharmaceutical composition includes at least one non-naturally occurring therapeutic or prophylactic agent.

The pharmaceutical composition can contain purified cis-suffruticosol D and/or trans-suffruticosol D, or it can contain a partially purified plant extract that contains cis-suffruticosol D and/or trans-suffruticosol D.

The formulations may be conveniently presented in unit dosage form and may be prepared by any of the methods well-known in the art of pharmacy. All methods include the step of bringing the active agent into association with a pharmaceutical carrier. In general, the formulations are prepared by uniformly and intimately bringing the active compound into association with a liquid carrier, a finely divided solid carrier, or both, and then, if necessary, shaping the product into the desired formulations.

Formulations of the present invention suitable for oral administration can be presented as discrete units such as tablets, troches, capsules, lozenges, wafers, or cachets, each containing a predetermined amount of the active agent as a powder or granules, as liposomes, or as a solution or suspension in an aqueous liquor or non-aqueous liquid such as a syrup, an elixir, an emulsion, or a draught. The tablets, troches, pills, capsules, and the like can also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it can further contain a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, sugar, and the like. A syrup or elixir can contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent can be incorporated into sustained-release or controlled release preparations and devices.

Formulations suitable for parenteral administration conveniently include a sterile aqueous preparation of the active agent, or dispersions of sterile powders of the active agent, which are preferably isotonic with the blood of the recipient. Parenteral administration of cis-suffruticosol D and/or trans-suffruticosol D (e.g., through an I.V. drip) is one form of administration. Isotonic agents that can be included in the liquid preparation include sugars, buffers, and sodium chloride. Solutions of the active agent can be prepared in water, optionally mixed with a nontoxic surfactant. Dispersions of the active agent can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the active agent, preferably by filter sterilization. Preferred methods for preparing powders include vacuum drying and freeze drying of the sterile injectable solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the active agents over a prolonged period can be achieved by including agents for delaying, for example, aluminum monostearate and gelatin.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration can be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations. Topical formulations can be provided in the form of a bandage, wherein the formulation is incorporated into a gauze or other structure and brought into contact with the skin.

Administration of cis-Suffruticosol D and/or trans-Suffruticosol D

The active agents cis-suffruticosol D and/or trans-suffruticosol D and a synthetic derivative thereof can be administered to a subject alone or in a pharmaceutical composition that includes the active agent and a pharmaceutically acceptable carrier. The term “administered” encompasses administration of a prophylactically and/or therapeutically effective dose or amount of cis-suffruticosol D and/or trans-suffruticosol D, or derivative thereof, to a subject. The subject is preferably a mammal, more preferably a domestic or domesticated animal or human. The term “effective dose” or “effective amount” refers to a dose or amount that produces the effects for which it is administered, especially an anticancer effect. cis-Suffruticosol D and/or trans-suffruticosol D and/or derivatives thereof can be introduced into the subject systemically or locally, for example at the site of a tumor.

cis-Suffruticosol D and/or trans-suffruticosol D can be administered in a variety of routes, including orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. Local administration can include topical administration, administration by injection, or perfusion or bathing of an organ or tissue, for example.

The formulations can be administered as a single dose or in multiple doses. Useful dosages of the active agents can be determined by comparing their in vitro activity and the in vivo activity in animal models. Methods for extrapolation of effective dosages in mice, and other animals, to humans are known in the art.

A mixture of cis-suffruticosol D and trans-suffruticosol D can be administered to a subject. For example, the extracted, isolated, purified, or synthesized cis-suffruticosol D can be present in a mixture that also includes trans-suffruticosol D, such that cis-suffruticosol D is at least 50% of the total cis- and trans-suffruticosol D, more particularly at least 60%, 70%, 80%, 85%, 90%, 95%, or 99% of the total cis- and trans-suffruticosol D.

Alternatively, the cis-suffruticosol D administered to a subject can be substantially or completely free of trans-suffruticosol D, or the trans-suffruticosol D administered to a subject can be substantially or completely free of cis-suffruticosol D

The relative amounts of cis-suffruticosol D, trans-suffruticosol D, and total cis- and trans-suffruticosol D can be measured by high-performance liquid chromatography (HPLC).

Dosage levels of the active agent, including but not limited to cis-suffruticosol D and/or trans-suffruticosol D, in the pharmaceutical compositions of this invention can be varied so as to obtain an amount of the active agent which is effective to achieve the desired therapeutic response for a particular subject, composition, and mode of administration, without being toxic to the subject. The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the cis-suffruticosol D and/or trans-suffruticosol D, the age, sex, weight, condition, general health and prior medical history of the subject being treated, and like factors well known in the medical arts.

Dosages and dosing regimens that are suitable for resveratrol and other stilbenoids are likewise suitable for therapeutic or prophylactic administration of cis-suffruticosol D and/or trans-suffruticosol D. For example, purified cis-suffruticosol D and/or trans-suffruticosol D can be administered orally in an amount of between 10 mg and 100 mg per day, as a medication, nutritional supplement, or food additive. As another example, cis-suffruticosol D and/or trans-suffruticosol D can be administered in dosages ranging from 0.01 mg/kg to 10 mg/kg body weight, or higher; or in a form sufficient to provide a daily dosage of 0.03 mg/kg body weight to about 10 mg per/kg body weight of the subject to which it is to be administered. See, e.g., U.S. Pat. Publication No. 2008/0262081 for nutraceutical compositions, dosing information and methods relating to resveratrol that are equally applicable to cis-suffruticosol D and/or trans-suffruticosol D.

cis-suffruticosol D and/or trans-suffruticosol D can also be administered as an extract obtained from a plant source, such as a seed. Dosages and dosing regimens that are suitable for melinjo seed extract and other seed extracts are likewise suitable for therapeutic prophylactic administration of plant extracts containing cis-suffruticosol D and/or trans-suffruticosol D. For example, between 20 and 1000 mg/day can be administered as a powdered extract in loose, capsule or tablet form. See, e.g., Konno et al., Evid. Based Complement Alternat. Med., 2013:589169 (2013); Tani et al., J. Agric. Food Chem., 62(8):1999-2007 (2014).

A physician having ordinary skill in the art can readily determine and prescribe the effective amount of the pharmaceutical composition required. For example, the physician could start doses of the cis-suffruticosol D and/or trans-suffruticosol D of the invention employed in the pharmaceutical composition at levels lower than that required in order to achieve the desired therapeutic effect and gradually increase the dosage until the desired effect is achieved.

### Methods of Treatment

The efficacy of cis-suffruticosol D and/or trans-suffruticosol D against tumor cell lines was unknown prior to the present work. Example 1 demonstrates that cis-suffruticosol D and/or trans-suffruticosol D have significant anticancer activity.

The invention therefore provides a method for treating or preventing cancer or a precancerous condition in a subject, and/or inhibiting or reversing tumor growth in a subject, by administering to a subject a composition that contains cis-suffruticosol D and/or trans-suffruticosol D and/or a derivative thereof, in an amount effective to treat or prevent the cancer or precancerous condition, or inhibit or reverse growth of the tumor. Administration of the composition can be performed before, during, or after a subject develops cancer, a precancerous condition or a tumor.

In one embodiment, the method is a therapeutic method for treating a subject suffering from a cancer or a precancerous condition by administering cis-suffruticosol D and/or trans-suffruticosol D and/or derivatives thereof to the subject in an amount effective to treat the cancer or precancerous condition. In another embodiment, the therapeutic method includes administering cis-suffruticosol D and/or trans-suffruticosol D and/or derivatives thereof to a subject who has a tumor, in an amount effective to inhibit, slow, or reverse growth of the tumor. Therapeutic treatment is initiated after the development of cancer, a precancerous condition, or a tumor. Treatment initiated after the development of cancer may result in decreasing the severity of the symptoms of one of the conditions, or completely removing the symptoms. cis-Suffruticosol D and/or trans-suffruticosol D can be introduced into the mammal either systemically or at the site of a cancer tumor.

cis-suffruticosol D and/or trans-suffruticosol D can also be administered prophylactically, e.g., as a chemopreventive agent, in an amount effective to prevent or delay the development of cancer or a precancerous condition in a subject. Treatment that is prophylactic, for instance, can be initiated before a subject develops cancer or manifests cancer symptoms. An example of a subject that is at particular risk of developing cancer is a person having a risk factor, such as a genetic marker, that is associated with the disease. Examples of genetic markers indicating a subject has a predisposition to develop certain cancers include alterations in the BRCA1 and/or BRCA2 genes (breast, prostate, or colon cancer) and HPC1 (prostate cancer).

The method of the invention can be used to treat a variety of cancerous or precancerous conditions, including tumors or dysplasia. A tumor can be a solid tumor, such as a carcinoma, a sarcoma, or a lymphoma, and can be present, for example, in the bone, brain, breast, cervix, larynx, lung, pancreas, prostate, skin, spine, stomach, or uterus. The cancer treated by the method of the invention can also be a blood cancer, such as a leukemia. The dysplasia can be an epithelia dysplasia. The tumor can made up of tumor cells, including lymphoid and myeloid cancers; multiple myeloma; cancers of the bone, breast, prostate, stomach, colon, pancreas, and thyroid; melanoma; head and neck squamous cell carcinoma; ovarian carcinoma; and cervical carcinoma.

Administration of cis-suffruticosol D and/or trans-suffruticosol D to treat or prevent cancer, a precancerous condition, or to inhibit or reverse tumor growth, can occur before, during, and/or after other treatments. Such combination therapy can involve the administration of cis-suffruticosol D and/or trans-suffruticosol D before, during and/or after the use of other anti-cancer agents, for example, chemotherapeutic agents or radiation or both. Examples of combination therapy may involve two or more therapeutic agents being administered concurrently, or being separately administered in an alternating or other periodic fashion, or being administered in succession over time. It is expected that cis-suffruticosol D and/or trans-suffruticosol D may potentiate the effects of cytokines, chemotherapeutic agents, or gamma radiation (see, e.g., Aggarwal et al., Anticancer Res. 24:2783-2840 (2004)). The administration of cis-suffruticosol D and/or trans-suffruticosol D can be separated in time from the administration of other anti-cancer agents by hours, days, or even weeks; alternatively, cis-suffruticosol D and/or trans-suffruticosol D can be administered concurrently with other anti-cancer agents, either together in the same composition or in separate compositions. Additionally or alternatively, the administration of cis-suffruticosol D and/or trans-suffruticosol D can be combined with other biologically active agents or modalities and/or non-drug therapies, such as, but not limited to, surgery. Additional biologically active agents that can be utilized with cis-suffruticosol D and/or trans-suffruticosol D in combination therapies include, without limitation, an antineoplastic agent, an antiangiogenic agent, a chemopreventive agent, an anti-inflammatory agent, a cytokine, a chemokine, a therapeutic antibody, an immunogen, an antigen, an adjuvant, or an antioxidant, an immunomodulatory compound, an analgesic, and a biologic compound.

Combination therapy is often used, for example, in the treatment of breast cancer, and can also be used prophylactically for persons at high risk of developing breast cancer. cis-Suffruticosol D and/or trans-suffruticosol D can advantageously be utilized in combination with any desired anti-cancer therapeutic agent. Illustrative chemotherapeutic agents that can be used in combination with cis-suffruticosol D and/or trans-suffruticosol D include, without limitation, anthracyclines (such as doxorubicin/Adriamycin® and epirubicin/Ellence®); taxanes (such as paclitaxel/Taxol® and docetaxel/Taxotere®); fluorouracil (5-FU); cyclophosphamide (Cytoxan®); carboplatin; trastuzumab (Herceptin®) and Pertuzumab (Perjeta®). cis-Suffruticosol D and/or trans-suffruticosol D can be substituted for, or used in addition to, any of the commonly used drug combinations for breast cancer. Examples of commonly used combinations (in which cis-suffruticosol D and/or trans-suffruticosol D can be substituted, or used in addition) used for early treatment of breast cancer include:

CAF (or FAC): cyclophosphamide, doxorubicin (Adriamycin), and 5-FU

TAC: docetaxel (Taxotere), doxorubicin (Adriamycin), and cyclophosphamide

AC→T: doxorubicin (Adriamycin) and cyclophosphamide followed by paclitaxel (Taxol) or docetaxel (Taxotere), or the reverse order, with the T (paclitaxel or docetaxel) given first, followed by AC, with carboplatin optionally added to paclitaxel FEC→T, 5-FU, epirubicin, and cyclophosphamide followed by docetaxel (Taxotere) or paclitaxel (Taxol), or the reverse order, with carboplatin optionally added to paclitaxel

TC: docetaxel (Taxotere) and cyclophosphamide

TCH: docetaxel, carboplatin, and trastuzumab (Herceptin)

CMF: cyclophosphamide (Cytoxan®), methotrexate, and 5-fluorouracil (fluorouracil, 5-FU)

A→CMF: doxorubicin (Adriamycin), followed by CMF

EC: epirubicin (Ellence) and cyclophosphamide

AC: doxorubicin (Adriamycin) and cyclophosphamide

Examples of chemotherapeutic agents useful in treating women with advanced breast cancer, which can be used in combination with cis-suffruticosol D and/or trans-suffruticosol D, include docetaxel, paclitaxel, platinum agents (cisplatin, carboplatin), vinorelbine (Navelbine®), capecitabine (Xeloda®), liposomal doxorubicin (Doxil®), gemcitabine (Gemzar®), mitoxantrone, ixabepilone (Ixempra®), albumin-bound paclitaxel (nab-paclitaxel or Abraxane®) and eribulin (Halaven®).

Also, cis-suffruticosol D and/or trans-suffruticosol D can be administered to a subject who has recovered from cancer, preferably breast cancer, as a maintenance medication after remission has been achieved, to help maintain remission.

### Compositions and Methods for Veterinary Use

Any of the compositions or methods described herein that include cis-suffruticosol D and/or trans-suffruticosol D, or variant, derivative, analog, modification, or conjugate thereof, can be used in veterinary applications. Veterinary uses in domestic or domesticated animals (including small animals such as cats, dogs, and other pets, as well as large animals such as cows, horses, pigs, and other livestock), as well as wild animals (e.g., animals housed in zoos) to treat or prevent cancer or a precancerous conditions are examples of contemplated applications.

### Kits

The invention further includes a kit that contains cis-suffruticosol D and/or trans-suffruticosol D, or derivatives thereof, together with instructions for use. In some embodiments, the instructions for use provide instructions for use in the treatment or prevention of cancer, a precancerous condition, or a tumor. Optionally, the kit includes a pharmaceutically acceptable carrier. The carrier may be separately provided, or it may be present in a composition that includes cis-suffruticosol D and/or trans-suffruticosol D, and/or a derivative thereof. Optionally, the kit may further include one or more additional active agents which can be co-administered with the cis-suffruticosol D and/or trans-suffruticosol D, and/or derivatives thereof. The one or more active agents may have cumulative or complementary activities, as described in more detail elsewhere herein.

### Nutritional Supplement and Food Additive

cis-Suffruticosol D and/or trans-suffruticosol D can be packaged as a nutritional, health or dietary supplement (e.g., in pill or capsule form). The supplement can be optionally formulated for sensitive populations, and thus can be gluten-free, wheat-free, dairy-free, sugar-free and/or free of preservatives. Additionally, cis-suffruticosol D and/or trans-suffruticosol D can be added to a food product to yield what is commonly referred to as a “nutraceutical” food or “functional” food. Foods to which cis-suffruticosol D and/or trans-suffruticosol D can be added include, without limitation, animal feed, cereals, soups, beverages, yogurts, cottage cheeses, and other milk products, oils including hydrogenated or partially hydrogenated oils. In one embodiment, cis-suffruticosol D and/or trans-suffruticosol D and/or a derivative thereof is formulated as a nutritional supplement or food additive for domestic or domesticated animals, such as pets or livestock. Conveniently, cis-suffruticosol D and/or trans-suffruticosol D and/or a derivative thereof can be incorporated into animal feed such as fodder and kibble.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### In Vitro Antitumor Effects of cis- and trans-Suffruticosol D

### Abstract

Naturally derived stilbenes have been shown to elicit cytotoxic, anti-steroidal, anti-mutagenic, anti-oxidative, anti-inflammatory, and anti-tumor bioactivities. Previous phytochemical studies revealed that the seeds of Paeonia suffruticosa are rich in natural stilbenes. In this study the anti-tumor effects and mechanism of action of the oligostilbene isomers, cis- and trans-suffruticosol D, isolated from the seeds of P. suffruticosa were examined. cis- and trans-Suffruticosol D exhibited remarkable cytotoxicity against the human cancer cell lines A549 (lung), BT20 (breast), MCF7 (breast), and U2OS (osteosarcoma), but showed significantly less toxicity to the normal human cell line HMEC. cis- and trans-Suffruticosol D exerted their anti-tumor effects by provoking oxidative stress, stimulating apoptosis, decreasing the mitochondrial membrane potential, inhibiting cell motility, and blocking the NF-κB pathway in human lung cancer cells. In addition, the respective bioefficacy was evaluated and trans-suffruticosol D was found to be more potent than cis-suffruticosol D. Collectively, the results suggest that cis- and trans-suffruticosol D could be promising chemotherapeutic agents against cancer. See Almosnid et al. (2016) Int. J. of Oncol. 48(2):646-656, Epub Nov. 26, 2015.

### Introduction

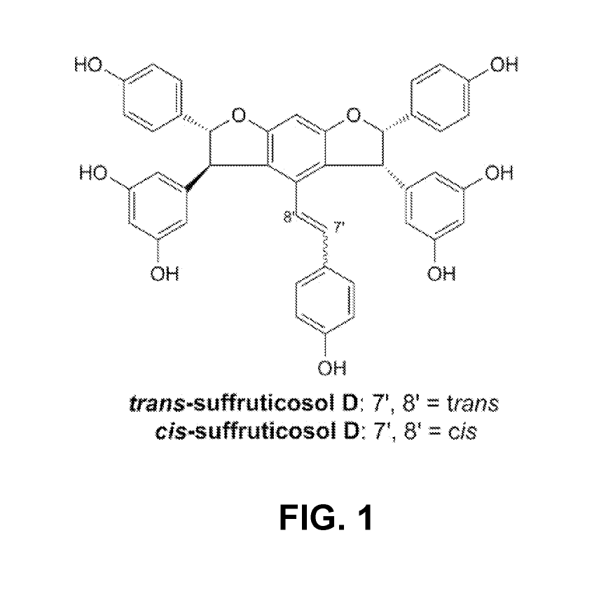

Previously, two novel stilbenes, cis- and trans-suffruticosol D, were extracted from the seeds of Paeonia (He et al., 2010a). The two chemicals have similar structures as the mass fragmentation pattern of trans-suffruticosol D was very similar to cis-suffruticosol D, with cis-suffruticosol D varying only from trans-suffruticosol D in its olefinic hydrogen signal (FIG. 1). In this study, the antitumor activities of cis- and trans-suffruticosol D were investigated and how these two chemicals act against cancer cells in vitro was examined.

### Methods and Materials

Plant material. The seeds of P. suffruticosa were collected in Tongling, Anhui province, P. R. China, and identified in September 2012. A voucher specimen (2012001) has been deposited in the Seed Resource Bank of the Institute of Medicinal Plant Development, Chinese Academy of Medical Sciences and Peking Union Medical College.

Extraction and isolation. Cis- and trans-suffruticosol D were extracted and isolated from the dried seeds of P. suffruticosa (1.2 kg) using procedures described in He et al. (2010a). Compounds were re-suspended in dimethyl sulfoxide (DMSO) (Sigma) to yield a concentration of 10 mM and stored at 4° C.

Cell culture. Four human cancer cell lines including A549 (lung carcinoma), BT20 (estrogen receptor-negative human breast adenocarcinoma), MCF-7 (estrogen receptor-positive human breast adenocarcinoma) and U2OS (human osteosarcoma) were purchased from American Type Culture Collection (ATCC). An A549 cell line that stably expresses Green Fluorescent Protein (GFP) was purchased from Cell BioLabs Inc. (San Diego, Calif., USA). A549, A549-GFP and BT20 cells were cultured in RPMI 1640 media (Sigma-Aldrich, St. Louis, Mo., USA), MCF7 cells were cultured in DMEM medium (ATCC, Manassas, Va., USA), and U20S cells were cultured in McCoy's 5A medium (ATCC, Manassas, Va., USA). As a control, HMEC cells (primary human mammary breast epithelial cells) were purchased from ATCC and cultured in McCoy's 5A medium. All medium contained 10% FBS (Sigma-Aldrich, St. Louis, Mo., USA) and 1% streptomycin and penicillin (Sigma-Aldrich, St. Louis, Mo., USA). These cells were incubated in a humid environment with 5% CO₂ at 37° C.

Cell proliferation assay. The resazurin reduction reagent AlamarBlue (Invitrogen, Frederick, Md., USA) was used to evaluate the cytotoxicity of the compounds. Cells were plated at a density of 5×10³ cells per well in 96-well microplates with 100 μl culture medium, and were allowed to attach for 16 h prior to treatment. Next, all the medium was replaced with medium containing the cis- or trans-suffruticosol D compounds at seven different concentrations: 320, 100, 32, 10, 3.2, 1.0, and 0.32 μM. The cells were placed in an incubator for 48 h at 37° C. Cells that were treated with medium-containing vehicle (1% DMSO) only serve as negative control. Subsequently, AlamarBlue solution was added to the medium and the cells were incubated in the CO₂ incubator for 1 h. The fluorescent intensity change of the dye was measured at Ex 555 nm and Em 590 nm using a plate reader (Molecular Devices, Sunnyvale, Calif., USA). The cytotoxicity was examined by determining by IC₅₀, the dose that inhibited 50% of cell growth, using GraphPad Prism software (GraphPad Software, La Jolla, Calif., USA).

Apoptosis assay. The FlowCellect Annexin Red Kit (EMD Millipore, Billerica, Mass., USA) was used to determine the apoptosis rate in A549 cells according to the manufacturer's instructions. Briefly, A549 cells were plated in 96-well plates. After a 24 h treatment with cis- or trans-suffruticosol D at concentrations of 100, 32, and 10 μM, the floating and attached cells were collected for analysis. The cells were centrifuged at 700×g for 7 min and were resuspended in 100 μL Assay Buffer (EMD Millipore, Billerica, Mass., USA). Afterwards, the cells were stained with annexin-V for 15 min and 7-amino-actinomycin D (7-AAD) for 5 min, and examined with a Guava EasyCyte Flow Cytometer (EMD Millipore, Billerica, Mass., USA). Data was analyzed using Guava InCyte software.

Apoptosis antibody array. The Human Apoptosis Antibody Array Kit (RayBiotech, Inc., Norcross, Ga. USA) was used to evaluate apoptotic protein expression according to the manufacturer's instructions. A549 cells were plated at 8,000 cells/well intensity in a 96-well plate and then treated with cis or trans-suffruticosol D at a concentration of 50 μM for 6 h. The cells were lysed in lysis buffer with protease inhibitors. The cell lysates were concentrated using a protein concentration column (EMD Millipore, Billerica, Mass., USA) to a total protein concentration of 2 mg/ml. The samples were then diluted 10 fold with assay buffer and incubated with an array membrane for 2 h at room temperature, and washed with washing buffer five times. Subsequently, the cocktail of biotin-conjugated antibody mix was added to the membrane and incubated overnight at 4° C. The samples were then incubated with HRP-conjugated streptavidin for 2 h at room temperature and chemiluminescence substrate was used to detect the signal. Image Studio software (LI-COR Biotechnology) was used to quantify the intensity of each array dot and then normalized to the internal control.

Oxidative stress assay. The Hitkit oxidative stress kit (Thermo Scientific, Waltham, Mass., USA) was used to determine the generation of reactive oxygen species (ROS) according to the manufacturer's instructions. Briefly, A549 cells were treated with cis- or trans-suffruticosol D for 24 h, fixed with warm 37% formaldehyde and stained with Hoechst and dihydroethidium (DHE) dye for 30 min at 37° C. with 5% CO₂. Doxorubicin (DOX) at 1 μM concentration was used as a positive control and cells treated with vehicle only were used as negative control. ROS generation in the nuclei was indicated by the production of the fluorescent ethidium, and assessed by measuring the nuclear fluorescent intensity using an ArrayScan VTI High-content screening (HCS) reader (Thermo Scientific, Waltham, Mass., USA). Images were acquired and data was analyzed by vHCS Scan software.

Cell motility assay. A 96-well collagen plate (Corning, N.Y., USA) was coated with blue fluorescent beads (Life Technologies, Eugene, Oreg., USA) as follows. The beads were centrifuged for 1 min at 14,000 g and washed twice with PBS, then 75 μl beads were added to each well of the 96-well collagen plate and incubated for 1 h at 37° C. The cells were seeded on the lawn of fluorescent beads and the sizes of the tracks generated by migrating cells were measured. After the plate was washed 5 times with PBS, A549-GFP cells were seeded at 500 cells/well in the coated plate and incubated for 1 h at 37° C. Subsequently the cells were treated with different concentrations of cis- or trans-suffruticosol D in medium containing 10% FBS for 18 h. Cells treated with serum-free medium serve as the negative control and cells treated with medium containing 10% FBS serve as the positive control. Cell tracks were imaged using an Arrayscan VTI HCS reader (Thermo Scientific, Waltham, Mass., USA) and the data was analyzed by vHCS Scan software. The mean of the full track area per cell for the test compound and the controls was calculated.

Multi-parameter cytotoxicity assay. HCS analysis was used to measure nuclear morphology, cell membrane permeability, and mitochondrial membrane potential changes, the three parameters associated with cytotoxicity. A549 cells were treated with different concentrations of cis- or trans-suffruticosol D for 24 h. The cells were then fixed and stained with a warm solution containing Hoechst dye, Membrane Permeability Dye, and Mitochondrial membrane Potential Dye (Thermo Scientific, Waltham, Mass., USA). Cells were imaged using an Arrayscan VTI HCS reader (Thermo Scientific, Waltham, Mass., USA). Data on nuclear size, cell permeability, and mitochondria membrane potential were collected and analyzed using vHCS Scan software.

Western blotting analysis. A549 cells were treated with 50 μM of cis- or trans-suffruticosol D for 3 h then incubated with 10 ng/mL of TNF-α for 30 min. Cells treated with the NF-κB inhibitor Bay11-7082 (10 μM) (Sigma-Aldrich) were used as a positive control, and cells treated with vehicle only were used as a negative control. After treatment, the cells were lysed using M-PER mammalian protein extraction reagent (Pierce PERBIO, Rockford, Ill.) containing proteinase and phosphatase inhibitors (Sigma-Aldrich, USA) and centrifuged at 13,000 rpm for 5 min at 4° C. A Pierce BCA protein assay kit (Thermo Fisher Scientific, Waltham, Mass., USA) was used to determined protein concentrations. Proteins were separated on a 4-20% Tris Glycine gel (Thermo Fisher Scientific, Waltham, Mass., USA), and electrophoretically transferred to a PVDF membrane. The following primary antibodies were used: phosphorylated-NF-κB p65, NF-κB p65 (Cell Signaling Technology, Danver, Mass.) and actin (Santa Cruz Biotechnology, Dallas, Tex.). The membrane was incubated with the primary antibodies at a 1:1000 concentration at 4° C. overnight. After washing with 1×PBS 5 times, the membrane was incubated for 2 h at room temperature with HRP linked anti-rabbit IgG secondary antibodies. Membranes were developed with chemiluminescent substrates (Thermo Fisher Scientific, Waltham, Mass., USA) and scanned with a chemiDoc MP imaging system (Bio-Rad, Hercules, Calif.).

NF-κB nuclear translocation assay. A Multiplexed NF-κB activation HCS Kit (Thermo Scientific, Waltham, Mass., USA) was used to assess NF-κB nuclear translocation. A549 cells were pre-treated with different concentrations of cis- or trans-suffruticosol D for 4 h, then 10 ng/mL of TNF-α was added to the cells for an additional 30 min. After treatment, cells were fixed and permeabilized prior to detection. NF-κB distribution was detected by adding NF-κB p65 primary antibodies and then staining with a secondary antibody conjugated with DyLight 549 and Hoechst dye (Thermo Scientific, Waltham, Mass., USA). Cells treated with medium containing only the vehicle were used as negative control, and cells treated with 25 ng/mL TNF-α were used as a positive control. Cells were imaged using an Arrayscan VTI HCS reader (Thermo Scientific, Waltham, Mass., USA). Data on the mean difference of NF-κB fluorescent intensity between the nuclear and cytoplasmic areas were collected and analyzed by vHCS Scan software.

### Results

Cytotoxicity of cis- and trans-Suffruticosol D in Lung, Breast, and Bone Cancer Cells.

After 48 h treatment, both cis- and trans-suffruticosol D showed significant cytotoxic effects against A549 (lung), BT20 (breast), MCF7 (breast) and U2OS (osteosarcoma) cancer cell lines. IC₅₀ values for cis- and trans-suffruticosol D against these cancer cells ranged from 9.93 to 46.79 μM as shown in Table 1. Interestingly, we observed that trans-suffruticosol D had lower IC₅₀ values (9.93-15.84 μM) than cis-suffruticosol D (13.42-46.79 μM) in all four cancer cell lines. In addition, both cis- and trans-suffruticosol D showed notably weaker cytotoxicity against normal breast epithelial cells HMEC (IC₅₀ values of 146.3 and 269.5 μM, respectively). The selectivity of cis- and trans-suffruticosol D ranged from 9.2-14.7 and from 5.8-20 fold, respectively (Table 1).

Table 1. IC₅₀ values of cis- and trans-suffruticosol D in selected cancer and normal cell lines. Cells were treated with various concentrations of cis- or trans-suffruticosol D for 48 h, and the viability of cells was evaluated with AlamarBlue dye.

TABLE 1

| | trans-SD | | cis-SD | |
|---|---|---|---|---|
| | IC₅₀ (μM) | Selectivity | IC₅₀ (μM) | Selectivity |
| A549 | 11.9 ± 1.2 | 12.3 | 17.1 ± 1.0 | 15.8 |
| BT20 | 9.9 ± 3.8 | 14.8 | 13.4 ± 2.5 | 20 |
| MCF-7 | 15.8 ± 1.6 | 9.3 | 46.8 ± 3.3 | 5.8 |
| U2OS | 11.3± 2.3 | 12.9 | 24.6 ± 4.4 | 11 |
| HMEC | 146.3 ± 2.7 | — | 269.5 ± 2.2 | — |

cis- and trans-Suffruticosol D Induce Apoptosis in A549 Lung Cancer Cells.

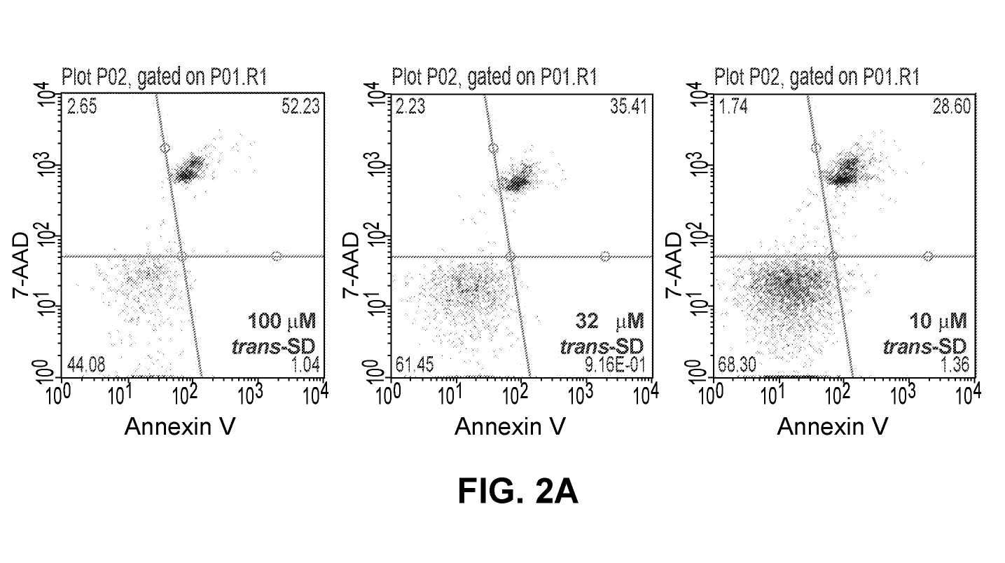

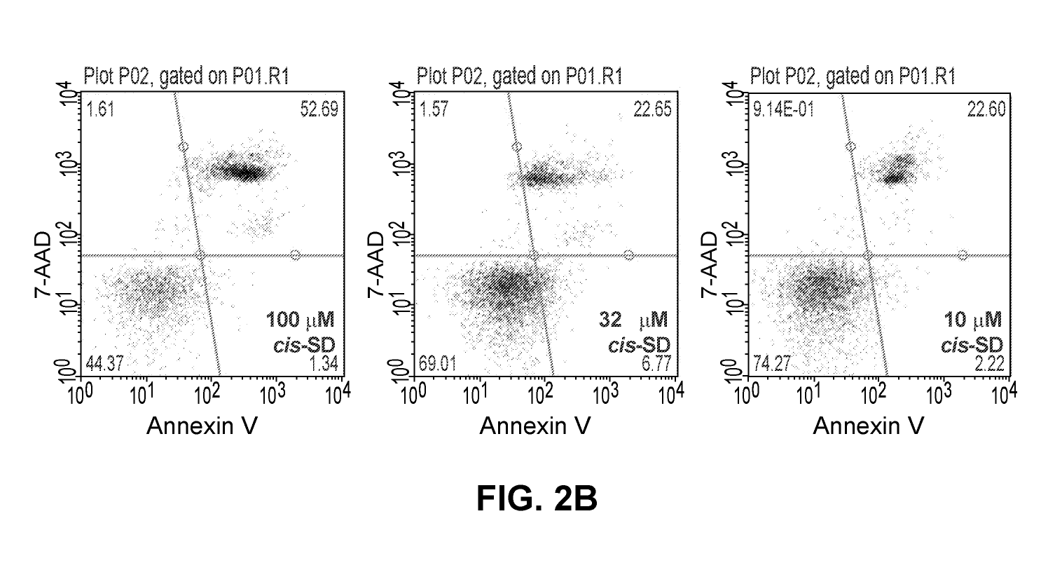

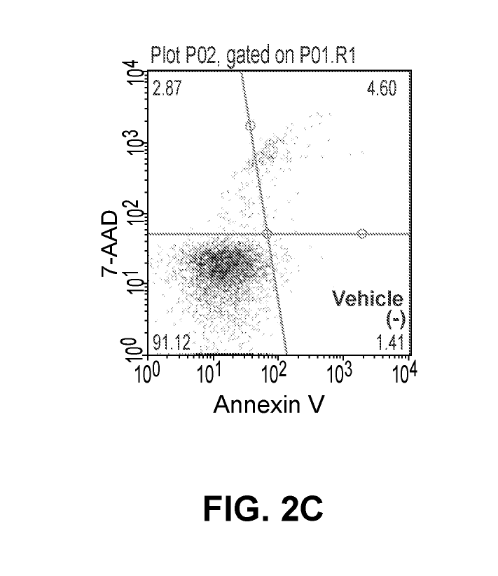

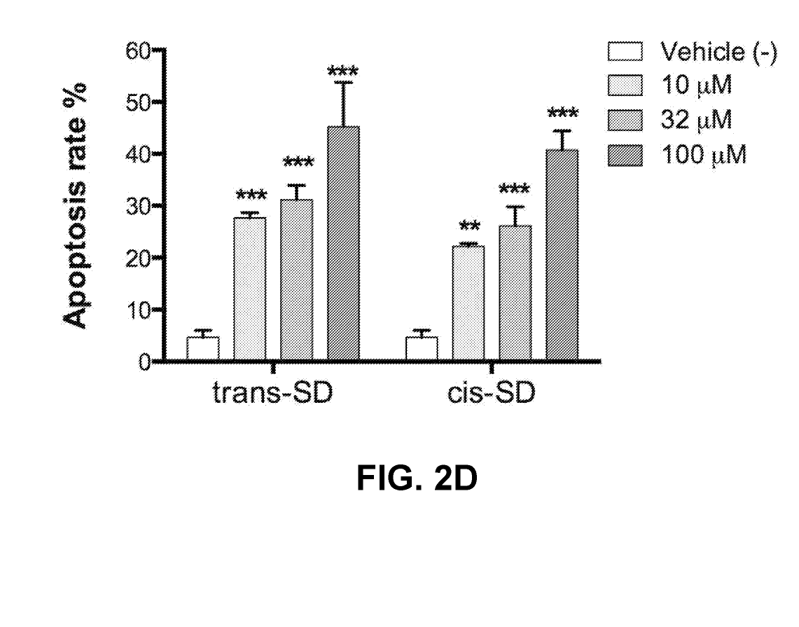

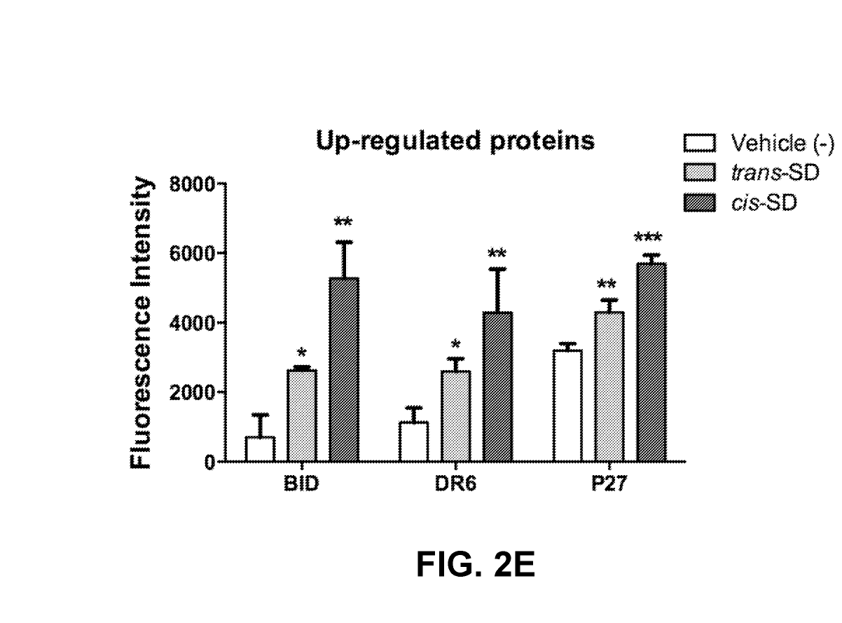

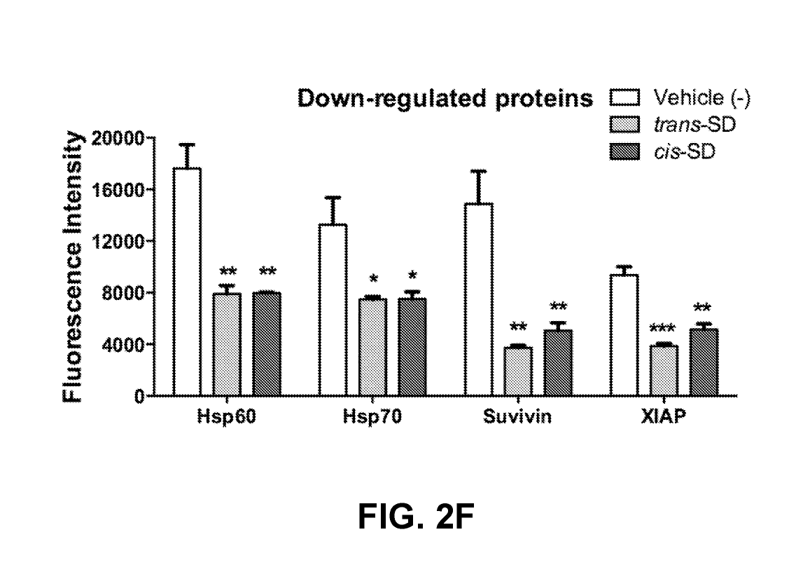

To find out whether the cytotoxic properties of cis- and trans-suffruticosol D were due to induction of apoptosis, we conducted an apoptosis assay using A549 cells treated with cis- or trans-suffruticosol D. Following 24 h treatment, both compounds showed significant apoptosis induction at a wide range of concentrations compared with the non-treated cells (*P<0.05, **P<0.01, or ***P<0.001) and the apoptotic effects were concentration-dependent (FIG. 2A-D). trans-suffruticosol D induced 30.1%, 39.8%, and 41.9% of A549 cells into apoptosis at concentrations of 10, 32, and 100 μM, respectively. cis-Suffruticosol D induced 22.2%, 27.1%, and 45.3% of A549 cells into apoptosis at concentrations of 10, 32, and 100 μM, respectively.

Next, we performed an apoptotic protein array analysis to investigate the effect of cis- and trans-suffruticosol D on apoptotic proteins. Two proteins from the inhibitor of apoptosis proteins family (IAPs), X-linked inhibitor of apoptosis protein (XIAP) and survivin, as well as the heat shock proteins Hsp60 and Hsp70, showed significant down regulation after treatment by cis- and trans-suffruticosol D (FIG. 2F). Meanwhile, death receptor 6 (DR6), also known as Tumor necrosis factor receptor superfamily member 21 (TNFRSF21); the cyclin-dependent kinase inhibitor 1B (p27^{Kip1}); and the BH3 interacting-domain death agonist (BID) were up-regulated by both cis- and trans-suffruticosol D (FIG. 2E).

cis- and trans-Suffruticosol D Induce ROS Generation in A549 Lung Cancer Cells

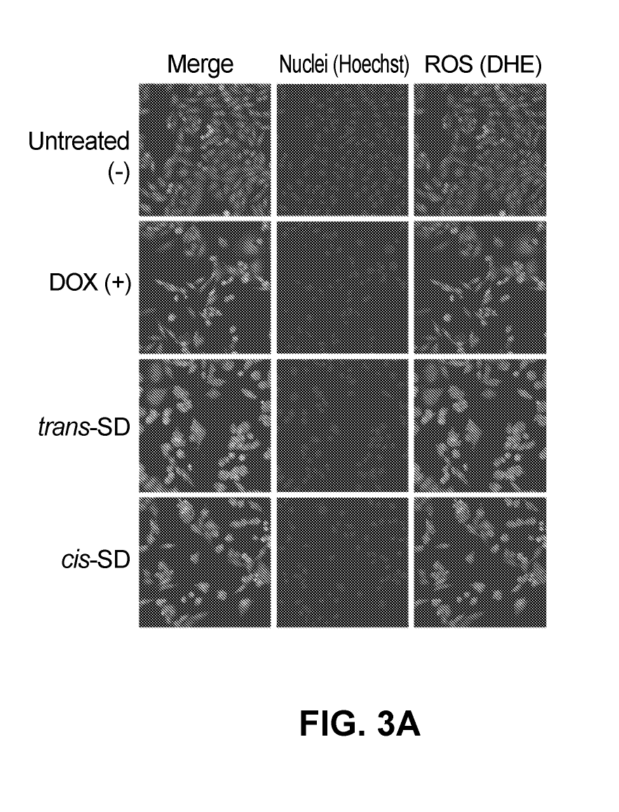

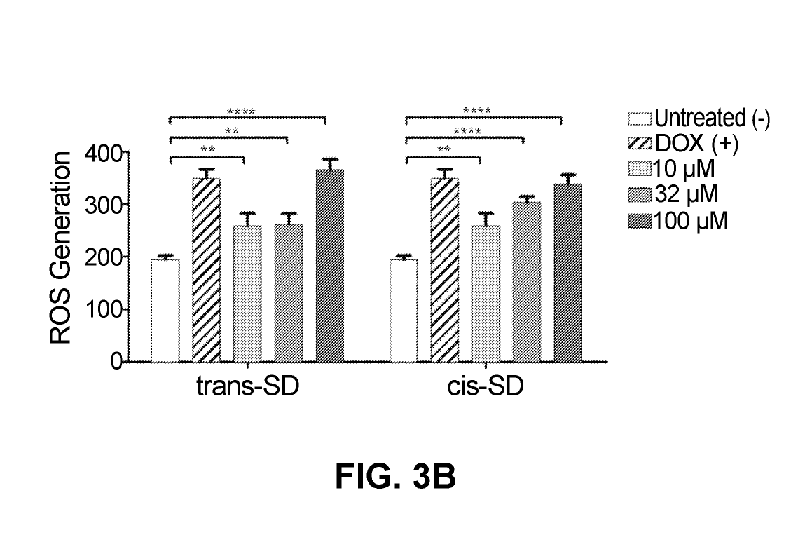

We examined the cellular ROS levels in A549 cells to determine whether cis- and trans-suffruticosol D induced oxidative stress. As shown in FIG. 3A, both cis- and trans-suffruticosol D converted non-fluorescent DHE to fluorescent ethidium, which binds to DNA, suggesting they induced ROS generation in A549 cells. Quantitative data showed both compounds significantly induced ROS generation in a concentration-dependent manner (**P<0.01, ***P<0.001, or ****P<0.0001). After treatment for 24 h, trans-suffruticosol increased the ROS levels by 32.8%, 34.6%, and 87.2% at concentrations of 10, 32, and 100 μM, respectively, while cis-suffruticosol increased the ROS levels by 32.8%, 55.6%, and 73.1% at concentrations of 10, 32, and 100 μM, respectively, in A549 cells (FIG. 3B).

cis- snd trans-Suffruticosol D Inhibit Motility of A549 Lung Cancer Cells

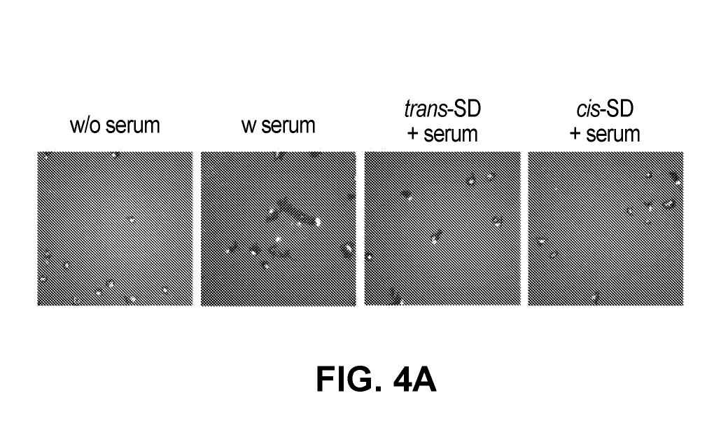

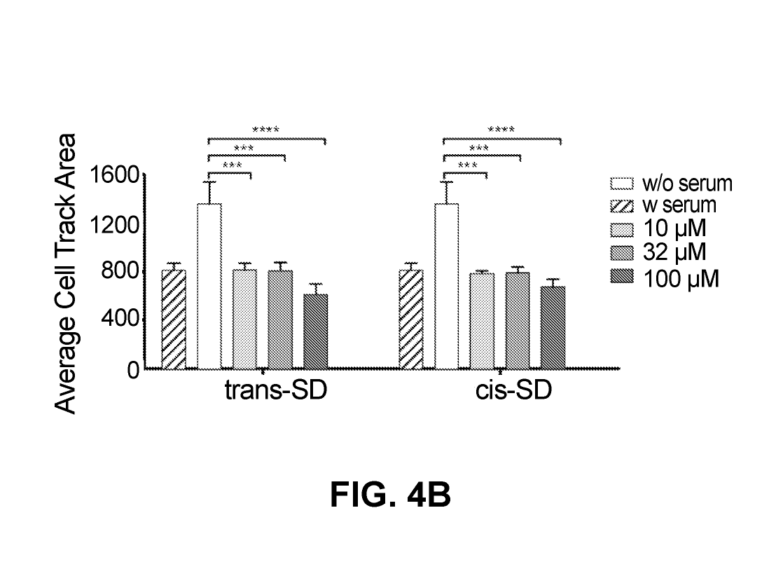

To test if cis- and trans-suffruticosol D affected cancer cell motility, we measured the area of the tracks generated by migrating cells after treatment, which is proportional to the magnitude of cell movement. As shown in FIG. 4A, A549 cells treated with cis- or trans-suffruticosol D in serum-containing medium showed less motility activity evidenced by a smaller track area per cell than the cells treated with serum-containing medium (complete medium) only. Both cis- and trans-suffruticosol D significantly inhibited cell movement at all the concentrations that were tested in A549 cells (***P<0.001 or ****P<0.0001) (FIG. 4B). trans-suffruticosol D decreased the A549 cell motility by 40.7%, 40.7%, and 54.9% at concentrations of 10, 32 and 100 μM, respectively, while cis-suffruticosol D decreased the A549 cell motility by 42.3%, 42.0%, and 50.4% at concentrations of 10, 32 and 100 μM, respectively.

### Multi-Parameter Cytotoxicity Assay

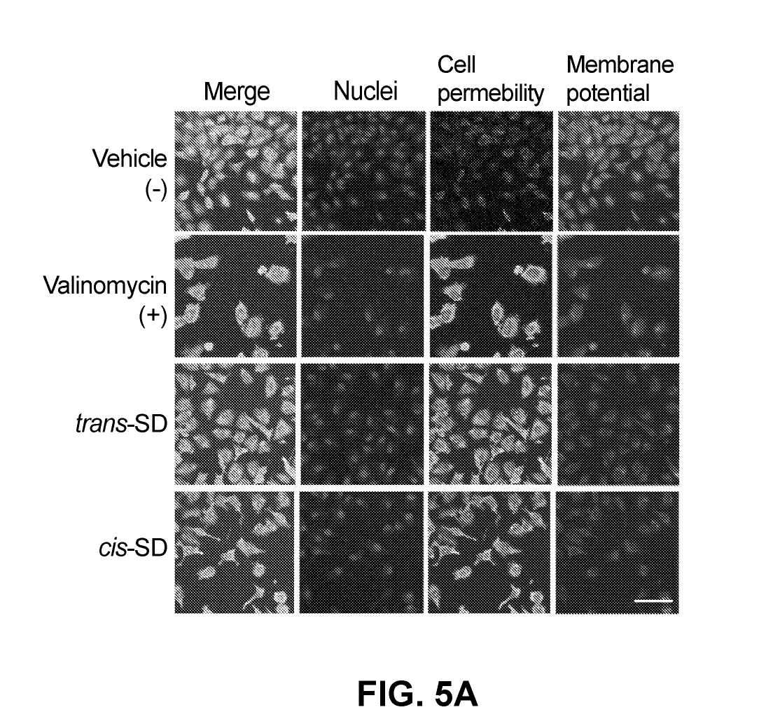

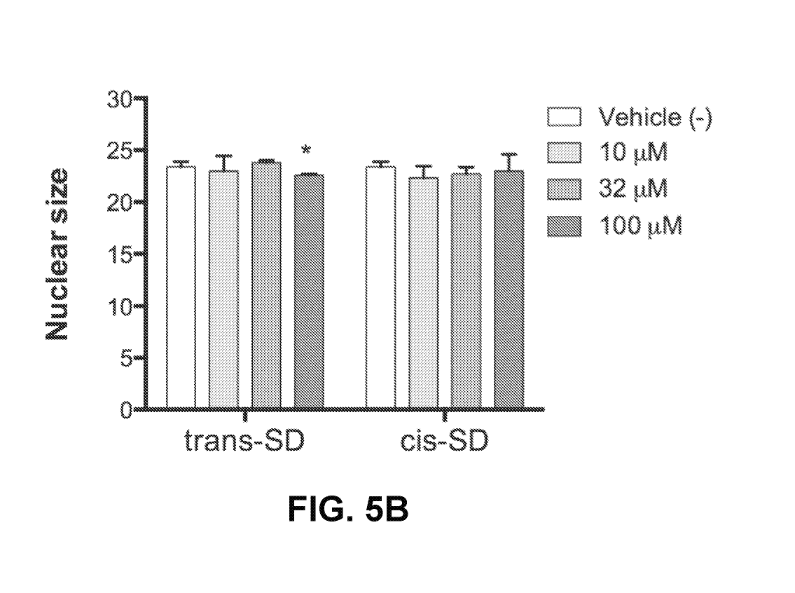

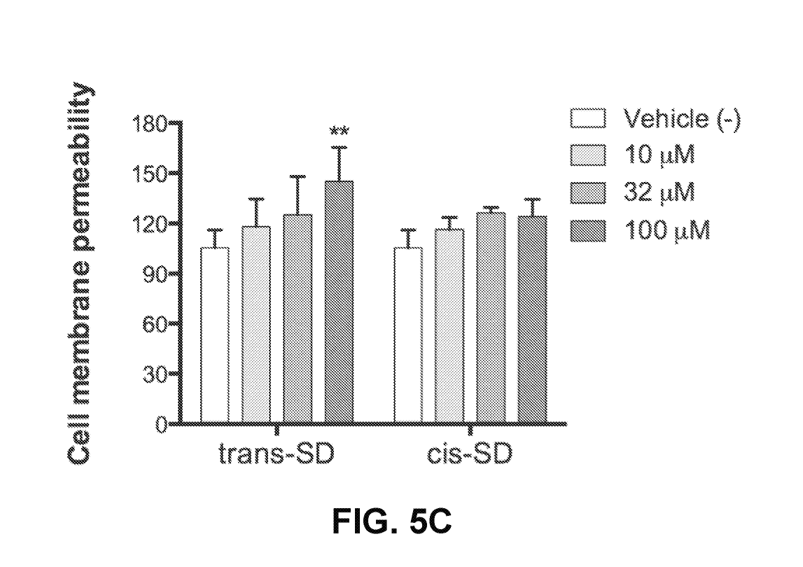

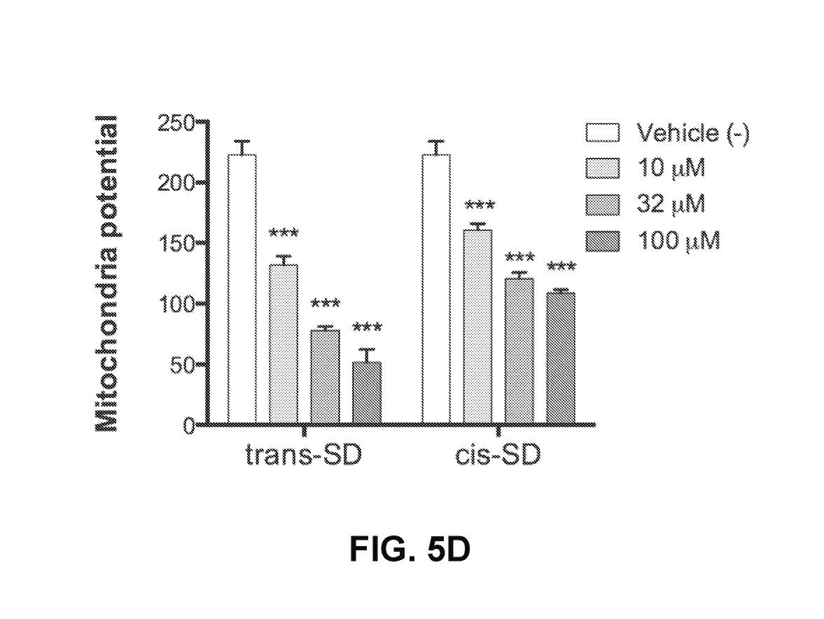

To determine the cytotoxic effect of cis- and trans-suffruticosol D in human lung cancer cells, we measured three cell health parameters, nuclear morphology, cell membrane permeability and mitochondrial membrane potential changes, using a high content screening (HCS) reader. As shown in FIG. 5A, in the mitochondrial potential channel, untreated A549 cells exhibited bright fluorescent intensity, indicating intact mitochondrial membranes. In comparison, in cells treated with cis- or trans-suffruticosol D, the fluorescent intensity of the dye was significantly decreased at all tested concentrations, indicating that cis- and trans-suffruticosol D induced a significant decrease of the mitochondrial membrane potential in A549 cells (***P<0.001) (FIG. 5D). We also observed nuclei shrinkage (FIG. 5B) and increased cell membrane permeability (FIG. 5C) in cells treated with a high-concentration (100 μM) of trans-suffruticosol D (*P<0.05 or **P<0.01). However, no significant change was detected in nuclear size and cell membrane permeability in cells treated with cis-suffruticosol D.

cis- and trans-Suffruticosol D Inhibit TNF-α-Induced NF-κB Activation

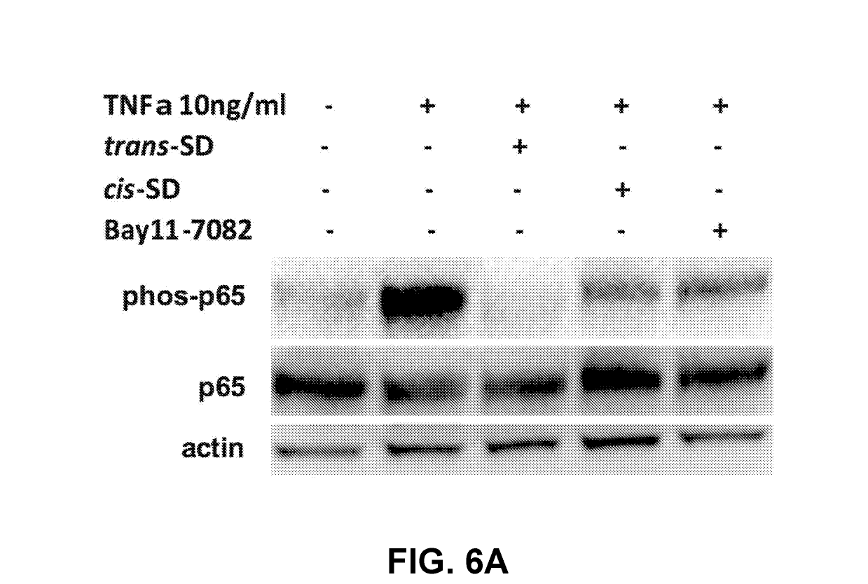

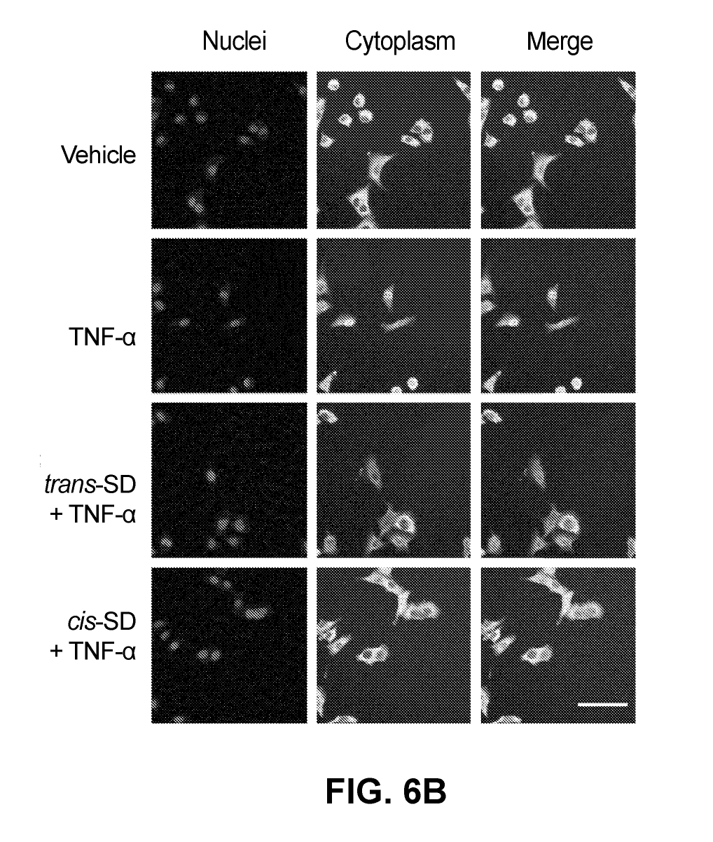

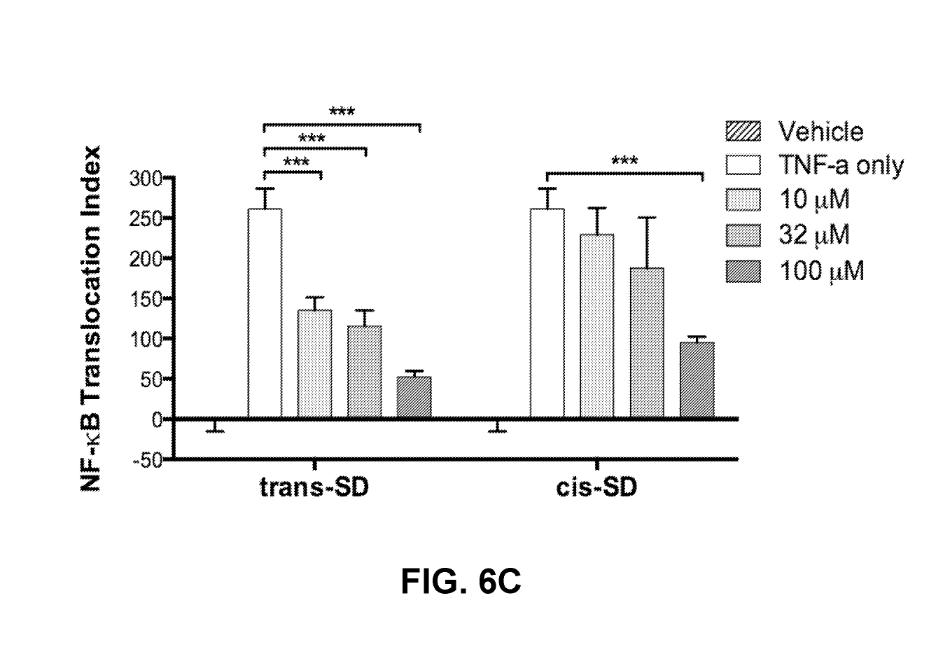

We performed western blot analysis to examine the effects of cis- and trans-suffruticosol D on the expression of NF-κB in A549 cells. As shown in FIG. 6A, upon TNF-α stimulation, overexpression of phosphorylated NF-κB p65 was detected, and the overexpression was significantly inhibited by cis- and trans-suffruticosol D. In trans-suffruticosol D treated cells, the expression of phosphorylated NF-κB p65 was almost completely blocked, and in cis-suffruticosol D treated cells, the expression of phosphorylated NF-κB p65 was blocked as effectively as the blockage caused by the Bay11-7082 inhibitor control.

Next, we used high content screening analysis to test whether cis- or trans-suffruticosol D could block NF-κB nuclear translocation in A549 cells. As shown in FIG. 6B, NF-κB fluorescent staining remained in the cytoplasmic area and no fluorescence was detected in the nuclear area in untreated cells; however, in cells treated with TNF-α, the NF-κB fluorescent staining was detected in the nuclear area, indicating that NF-κB was translocated from the cytoplasm to the nucleus. In A549 cells treated with cis- or trans-suffruticosol D, NF-κB fluorescent staining remained in the cytoplasm, suggesting that NF-κB translocation to the nucleus was blocked. Treatment with trans-suffruticosol D at all the tested concentrations, caused a significant inhibition of NF-κB activation (***P<0.001) (FIG. 6C). In contrast, treatment with cis-suffruticosol D only caused a significant inhibition of NF-κB at 100 μM (***P<0.001).

### Discussion

Oligostilbenes have been widely considered to be valuable resources of anti-tumor agents. Previously, two novel oligostilbenes, cis- and trans-suffruticosol D, were extracted from the seeds of P. suffruticosa, but their anti-tumor activities were not determined. In this study, we found that both of these oligostilbenes exhibited remarkable anti-proliferation activities against several types of cancer cell lines, and their cytotoxicity effects and related mechanisms were investigated.

trans-Suffruticosol D exhibited lower IC₅₀ values (9.93-20.8 μM) than cis-suffruticosol D (13.42-46.79 μM) in all of the cancer cell lines that were tested, indicating that trans-suffruticosol D is more cytotoxic than its cis isomer. Consistent with this conclusion, trans-suffruticosol D had stronger effects than cis-suffruticosol D on three cytotoxicity parameters, changes in nuclear size, cell membrane permeability and mitochondrial transmembrane potential. This observation is consistent with a previous report, which showed that trans-resveratrol had stronger cytotoxicity than its cis-isomer (Pettit et al., 2002). In addition, both chemicals showed selective cytotoxicity against cancer cell lines versus a normal cell line.

Cancer cells usually develop the ability to escape apoptosis, or programmed cell death, which is a homeostatic mechanism to maintain cell populations in the body (Kasibhatla & Tseng, 2003). Hence, targeting apoptotic induction has become an important strategy of anti-cancer therapies. It is commonly known that there are two apoptotic pathways, the extrinsic, or the death receptor pathway, and the intrinsic, or the mitochondrial pathway. Previous studies have shown that mitochondria play a critical role in apoptosis, especially in the intrinsic apoptosis pathways (Cheah et al., 2011; Ly, Grubb, & Lawen, 2003; Tedeschi, 1980). Mitochondria are the main source of ROS inside the cell, and increases in ROS production can damage the mitochondrial membrane and subsequently lead to the release of pro-apoptotic proteins and cytochrome c, thus activating the apoptotic pathway (Ozben, 2007; Sosa, 2013). In this study, we found that cis- and trans-suffruticosol D induced apoptosis in A549 lung cancer cells after 24 h treatment in a concentration-dependent manner. Both oligostilbenes significantly decreased the mitochondrial membrane potential in lung cancer cells, suggesting they might induce the mitochondrial apoptosis pathway. Since both chemicals significantly induced cellular ROS levels in lung cancer cells, it can be speculated that the excessive ROS induced by cis- and trans-suffruticosol D act as an apoptosis mediator by damaging the mitochondrial membrane, causing the release of the mitochondria's contents, which eventually leads to apoptosis. In addition, cis- and trans-suffruticosol D affected the expression of several key regulators involved in apoptosis; X-linked inhibitor of apoptosis protein (XIAP), survivin, heat shock protein 60 (Hsp60) and heat shock protein 70 (Hsp70) were down regulated, while BID (BH3 interacting-domain) death agonist, death receptor 6 (DR6) and cyclin-dependent kinase inhibitor 1B (p27^{KIP1}) were up regulated.

XIAP and survivin are known apoptosis inhibitors (Suzuki et al., 2001; Pa lidou et al., 2014) that prevent apoptosis by inhibiting caspases-3, -7, and -9 (Schimmer et al., 2006, Ryan et. al., 2009). Down regulation of XIAP or survivin has been demonstrated to inhibit the progression of cancer and increase the sensitivity of cancer cells to chemotherapeutic reagents (Hu et al., 2003; He et al., 2012; Oost et. al., 2004; Mita et al., 2008). Heat shock proteins Hsp60 and Hsp70 are chaperones that play essential roles in tumor cell survival and proliferation due to their ability to block both the intrinsic and extrinsic apoptosis pathways (Cappello et al., 2008, Murphy, 2013). BID is a pro-apoptotic member of the Bcl-2 protein family, and is a mediator of mitochondrial damage induced by caspase-8 (Luo et al., 1998). p27, the cyclin dependent kinase inhibitor, controls the cell cycle progression at G1 by preventing the activation of cyclin E-Cdk2 or cyclin D1-Cdk4 complexes (Yamamoto et. al., 1999, Nickeleit et al., 2007). DR6, also known as TNFRSF21, is a member of the death receptor family, which induces apoptosis in mammalian cells and its apoptotic function is inhibited by survivin (Kasof et al., 2001). Down regulation of XIAP, survivin, Hsp60 and Hsp70, as well as up-regulation of BID, DR6 and p27 by cis- and trans-suffruticosol D may at least partially contribute to the apoptotic effect of cis- and trans-suffruticosol D.

Tumor cells have the ability to migrate to surrounding tissues and organs through reorganization of the actin cytoskeleton (Yamazaki et al., 2005; Olson et al., 2009). Most of the fatality from tumors occurs when cells move from the initial organs where they originated (Wells et al., 2013). Therefore, control of cancer cell motility and migration is an essential issue in cancer treatment and represents a new opportunity for a potential tumor therapy (Levin, 2005). cis- and trans-Suffruticosol D significantly inhibited the mobility of lung cancer cells after treatment for 18 h at all the concentrations that were tested. Therefore, both chemicals exhibit therapeutic potential as an inhibitor of cancer cell mobility.

The NF-κB pathway is known to control cell growth and survival, and the transcription factor NF-κB has been found to be permanently activated in various tumors (Cheah et al., 2011; Monika et al., 2014). Activation of NF-κB in cancer cells is often associated with drug resistance as both radio- and chemo-therapies induce constitutive activation of the NF-κB pathway (Jin et al., 2008). Therefore a compound's ability to block the NF-κB pathway is important for the efficacy of cancer therapy (Monika et al., 2014; Nakanishi & Toi, 2005). In this study, we evaluated cis- and trans-suffruticosol D for their abilities to inhibit TNF-α induced NF-κB activation in lung cancer cells. After 4 h treatment both chemicals significantly blocked NF-κB p65 phosphorylation as well as NF-κB p65 translocation from the nucleus to the cytoplasm, suggesting they might act as an inhibitor of the NF-κB pathway. Since NF-κB affects the transcription of a number of anti-apoptotic proteins, including cellular inhibitor of apoptosis proteins (cIAP)s, XIAP, bcl-2, bcl-XL, FADD-like IL-1β-converting enzyme-inhibitory protein (c-FLIP) etc., blocking NF-κB nuclear translocation decreases the expression of anti-apoptotic proteins and subsequently promotes apoptosis. In addition, several studies have shown that an increase of ROS can block the NF-κB pathway by the inhibition of cytokines, such as TNF and IL-1 (Reuter et al., 2010). Because cis- and trans-suffruticosol D increased ROS generation in lung cancer cells, the block in the NF-κB pathway may be associated with the inhibition of the inducer cytokines by excessive ROS.

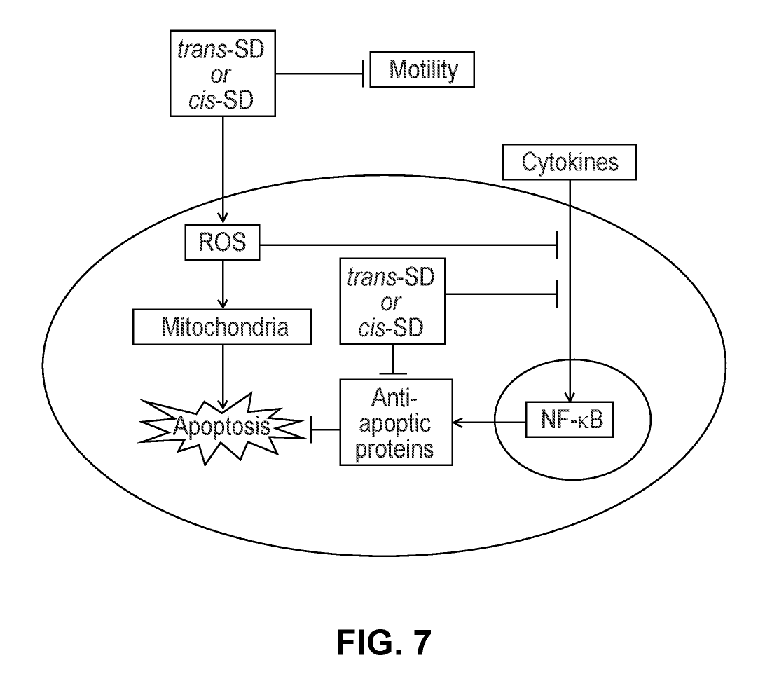

In conclusion, this study provides evidence that cis- and trans-suffruticosol D have promising antitumor activities. Both compounds selectively inhibited the growth of various cancer cells, induced apoptosis in A549 lung cancer cells, as well as inhibited A549 cell movement. The induction of apoptosis may be associated with ROS generation and inhibition of the NF-κB pathway. Collectively, our results suggest a potential mechanism for the cytotoxicity of cis- and trans-suffruticosol D. As shown in FIG. 7, in A549 lung cancer cells, cis- and trans-suffruticosol D trigger oxidative stress, which in turn leads to mitochondrial damage, blocks NF-κB activation and ultimately triggers apoptosis. Our findings suggest that both cis- and trans-suffruticosol D have promising chemotherapeutic potential for treating cancer.

### REFERENCES

Bo et al. (2002). A Selection of the Illustrated Chinese Anti-Cancer Herbal Medicines. Shanghai Science and Technology Literature Press, Shanghai.

Cai et al. (2011). cis-Ampelopsin E, a stilbene isolated from the seeds of Paeonia suffruticosa, inhibits lipopolysaccharide-stimulated nitric oxide production in RAW 264.7 macrophages via blockade of nuclear factor-kappa B signaling pathway. Biological and Pharmaceutical Bulletin, 34(9), 1501-1507.

Cai et al. (2004). Antioxidant activity and phenolic compounds of 112 traditional Chinese medicinal plants associated with anticancer. Life sciences, 74(17), 2157-2184.

Cappello et al. (2008). Hsp60 expression, new locations, functions and perspectives for cancer diagnosis and therapy. Cancer Biol Ther 7: 801-809.

Cheah et al. (2011). Panduratin a inhibits the growth of A549 cells through induction of apoptosis and inhibition of NF-KappaB translocation. Molecules, 16(3), 2583-2598.

Chinese Pharmacopoeia Commission. (2010), In: Chinese Pharmacopoeia. China Medical Scientific and Technological Press, Beijing, pp160-161.

Deavall et al. (2012). Drug-induced oxidative stress and toxicity. Journal of toxicology, 2012.

He at al. (2009). Preparative isolation and purification of antioxidative stilbene oligomers from Vitis chunganeniss using high-speed counter-current chromatography in stepwise elution mode. J Sep Sci 32: 2339-2345.

He et al. (2010a). Three new oligostilbenes from the seeds of Paeonia suffruticosa. Chemical and Pharmaceutical Bulletin, 58(6), 843-847.

He et al. (2010b). Phytochemical and biological studies of Paeoniaceae. Chemistry & Biodiversity, 7(4), 805-838.

He et al. (2012). HtrA1 sensitizes ovarian cancer cells to cisplatin-induced cytotoxicity by targeting XIAP for degradation. Int J Cancer 130: 1029-1035.

He et al. (2013). Simultaneous determination of ten stilbenes in the seeds of Paeonia species using HPLC-DAD. Journal of Liquid Chromatography & Related Technologies, 36(12), 1708-1724.

Hu et al. (2003). Antisense oligonucleotides targeting XIAP induce apoptosis and enhance chemotherapeutic activity against human lung cancer cells in vitro and in vivo. Clin Cancer Res 9: 2826-2836.

Hussain et al. (2009). Stilbene glycosides are natural product inhibitors of FGF-2-induced angiogenesis. BMC Cell Biol., 10(1), 30.

Ji et al. (1999) Pharmacological Action and Application of Anticancer Traditional Chinese Medicines Heilongjiang Science and Technology Publishing House, Ha'erbin, China

Jin et al. (2008). Potential biomarkers involving IKK/RelA signal in early stage non-small cell lung cancer. Cancer Science, 99(3), 582-589. doi: 10.1111/j.1349-7006.2007.00713.x

Jung et al. (2009). Synthesis, structural characterization and biological evaluation of novel stilbene derivatives as potential antimalarial agents. Chem Biol Drug Des 73: 346-354.

Kannan et al. (2000). Oxidative stress and apoptosis. Pathophysiology 7: 153-163.

Kasibhatla et al. (2003). Why target apoptosis in cancer treatment?. Molecular cancer therapeutics, 2(6), 573-580.

Kasof et al. (2001). Tumor necrosis factor-alpha induces the expression of DR6, a member of the TNF receptor family, through activation of NF-kappaB. Oncogene 20: 7965-7975.

Krajewski et al. (1999). Release of caspase-9 from mitochondria during neuronal apoptosis and cerebral ischemia. Proceedings of the National Academy of Sciences, 96(10), 5752-5757.

Lee et al. (2014). Stilbenes reduce Staphylococcus aureus hemolysis, biofilm formation, and virulence. Foodborne Pathog Dis 11: 710-717.

Levin (2005). Cancer therapy through control of cell migration. Current cancer drug targets, 5(7), 505-518.

Luo et al. (1998). Bid, a Bcl2 interacting protein, mediates cytochrome c release from mitochondria in response to activation of cell surface death receptors. Cell, 94(4), 481-490.

Ly et al. (2003). The mitochondrial membrane potential (Δψm) in apoptosis; an update. Apoptosis, 8(2), 115-128.

Mita et al. (2008). Survivin: Key regulator of mitosis and apoptosis and novel target for cancer therapeutics. Clin Cancer Res 14: 5000-5005.

Monika et al. (2014). Synthesis of lantadene analogs with marked in vitro inhibition of lung adenocarcinoma and TNF-α induced nuclear factor-kappa B (NF-κB) activation. Bioorganic & Medicinal Chemistry Letters, 24(16), 3814-3818.

Monks et al. (2002). Anti-tumour screening of Brazilian plants. Pharmaceutical biology, 40(8), 603-616.

Murphy (2013). The HSP70 family and cancer. Carcinogenesis 34:1181-1188, 2013.

Nakanishi et al. (2005). Nuclear factor-κB inhibitors as sensitizers to anticancer drugs. Nature Reviews Cancer, 5(4), 297-309.

Nickeleit et al. (2007). p27kip1: A target for tumor therapies? Cell Div 2: 13.

Niesen et al. (2013). Beyond resveratrol: A review of natural stilbenoids identified from 2009-2013. Journal of Berry Research, 3(4), 181-196.

Olson et al. (2009). The actin cytoskeleton in cancer cell motility. Clinical & experimental metastasis, 26(4), 273-287.

Oost et al. (2004). Discovery of potent antagonists of the antiapoptotic protein XIAP for the treatment of cancer, J Med Chem 47: 4417-4426.

Ozben (2007). Oxidative stress and apoptosis: impact on cancer therapy. Journal of pharmaceutical sciences, 96(9), 2181-2196.

Pavlidou et al. (2014) Association of survivin splice variants with prognosis and treatment of breast cancer, World J. Clin. Oncol., 5(5):883-94.

Parekh et al. (2009). A new dawn for the use of traditional Chinese medicine in cancer therapy. Molecular Cancer, 8(1), 21.

Pettit et al. (2002). Antineoplastic Agents. 465. Structural Modification of Resveratrol: Sodium Resverastatin Phosphate 1. Journal of medicinal chemistry, 45(12), 2534-2542.

Reuter et al. (2010). Oxidative stress, inflammation, and cancer: how are they linked?. Free Radical Biology and Medicine, 49(11), 1603-1616.

Ryan et al. (2009). Survivin: A new target for anti-cancer therapy. Cancer Treat Rev 35: 553-562.

Sangjun et al. (2009). Induction of cell cycle arrest in human MCF-7 breast cancer cells by cis-stilbene derivatives related to VIOXX®. Toxicology letters, 186(2), 115-122.

Savio et al. (2009). The resveratrol analogue 4,4′-dihydroxy-trans-stilbene inhibits cell proliferation with higher efficiency but different mechanism from resveratrol. The international journal of biochemistry & cell biology, 41(12), 2493-2502.

Schimmer et al. (2006). Targeting XIAP for the treatment of malignancy. Cell Death Differ 13: 179-188.

Shen et al. (2013). Stilbenoids. In: Natural Products. Springer, pp1901-1949.

Shukla et al. (2011). Resveratrol and cellular mechanisms of cancer prevention. Ann NY Acad Sci 1215: 1-8.

Simoni et al. (2009). Design, synthesis and biological evaluation of novel stilbene-based antitumor agents. Bioorganic & medicinal chemistry, 17(2), 512-522.

Sosa et al. (2013). Oxidative stress and cancer: an overview. Ageing research reviews, 12(1), 376-390.

Suzuki et al. (2001). X-linked inhibitor of apoptosis protein (XIAP) inhibits caspase-3 and -7 in distinct modes. J Biol Chem 276: 27058-27063.

Tedeschi (1980). The mitochondrial membrane potential. Biological Reviews, 55(2), 171-206.

Wells et al. (2013). Targeting tumor cell motility as a strategy against invasion and metastasis. Trends in pharmacological sciences, 34(5), 283-289.

Whitlock et al. (2012). The anticancer effects of resveratrol: Modulation of transcription factors. Nutr Cancer 64: 493-502.

Xu et al. (2000) Coloured Illustrations of Antitumor Chinese Traditional And Herbal Drugs (2nd) Fujian Science and Technology Publishing House, Fuzhou, China.

Yamamoto et al. (1999). Comparative effects of overexpression of p27Kip1 and p21Cip1/Waf1 on growth and differentiation in human colon carcinoma cells. Oncogene 18: 103-115.

Yamazaki et al. (2005). Regulation of cancer cell motility through actin reorganization. Cancer science, 96(7), 379-386.

Yuk et al. (2013). Profiling of neuraminidase inhibitory polyphenols from the seeds of Paeonia lactiflora. Food and Chemical Toxicology, 55, 144-149.

Zhang et al. (1998). Clinical Practice of Anticancer Traditional Chinese Medicines. People's Health Publishing House, Beijing.

The complete disclosures of all patents, patent applications including provisional patent applications, publications including patent publications and nonpatent publications, and electronically available material (e.g., GenBank amino acid and nucleotide sequence submissions) cited herein are incorporated by reference. The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

1. A method for treating or preventing cancer or a precancerous condition or for inhibiting the growth of a tumor in a subject, the method comprising:
administering to the subject a composition comprising an effective amount of cis-suffruticosol D, trans-suffruticosol D, or a combination thereof.

2. The method of claim 1, wherein the cancer is selected from bone cancer, brain cancer, breast cancer, cervical cancer, cancer of the larynx, lung cancer, pancreatic cancer, prostate cancer, skin cancer, cancer of the spine, stomach cancer, uterine cancer, or a blood cancer.

3. The method of claim 1, wherein the cancer is a metastatic cancer.

4. (canceled)

5. The method of claim 1, wherein the tumor comprises a solid tumor present in the bone, brain, breast, cervix, larynx, lung, pancreas, prostate, skin, spine, stomach, or uterus of the subject.

6. The method of claim 1, wherein the tumor is a fast growing tumor.

7. The method of claim 1, wherein the composition comprises cis- and trans-suffruticosol D.

8. The method of claim 1, wherein trans-suffruticosol D is at least 90% of the total suffruticosol D in the composition.

9. The method of claim 1, wherein the composition comprises an extract prepared from Paeonia sulfruticosa seeds.

10. The method of claim 1, wherein the composition further comprises a pharmaceutically acceptable carrier.

11. The method of claim 1, wherein the composition further comprises a non-naturally occurring therapeutic agent.

12. The method of claim 11, wherein the therapeutic agent comprises at least one of a cytokine, a chemokine, a therapeutic antibody, an adjuvant, an antioxidant, or a chemotherapeutic agent.

13. (canceled)

14. A pharmaceutical composition comprising:
cis-suffruticosol D, trans-suffruticosol D, or a combination thereof; and
a pharmaceutically acceptable carrier.

15. The pharmaceutical composition of claim 14, comprising:
a first active agent comprising cis-suffruticosol D, trans-suffruticosol D, or a combination thereof; and
a second active agent comprising at least one compound selected from the group consisting of an anticancer agent, antiangiogenic agent, a chemopreventive agent, an anti-inflammatory agent, a cytokine, a chemokine, a therapeutic antibody, an immunogen, an antigen, an adjuvant, or an antioxidant, an immunomodulatory compound, an analgesic, a biologic compound, an antineoplastic agent, and a chemotherapeutic agent.

16. The pharmaceutical composition of claim 15, wherein the second active agent is a non-naturally occurring compound.

17. The pharmaceutical composition of claim 15 wherein the second active agent is selected from the group consisting of doxorubicin (Adriamycin®), epirubicin (Ellence®), paclitaxel (Taxol®) docetaxel (Taxotere®), fluorouracil (5-FU), cyclophosphamide (Cytoxan®), trastuzumab (Herceptin®), Pertuzumab (Perjeta®), methotrexate, cisplatin, carboplatin, vinorelbine (Navelbine®), Capecitabine (Xeloda®), liposomal doxorubicin (Doxil®), gemcitabine (Gemzar®), mitoxantrone, ixabepilone (Ixempra®), albumin-bound paclitaxel (nab-paclitaxel or Abraxane®) and Eribulin (Halaven®).

18. The pharmaceutical composition of claim 14 in a controlled release formulation.

19. A dietary supplement or nutraceutical composition comprising cis-suffruticosol D, trans-suffruticosol D, or a combination thereof.

20. The dietary supplement of claim 19 formulated as animal feed, fodder or kibble.

21. (canceled)

22. The nutraceutical composition of claim 19 comprising a food selected from the group consisting a cereal, a beverage, a milk product, an oil and a soup.

23.-31. (canceled)
